SEQUENCE LISTING (36 sequences)

SEQ ID NO: 1 - DNA, 41 bp, Homo sapiens
tgagagctgattattttccarttaacaaagggtaggaagtt

SEQ ID NO: 2 - DNA, 41 bp, Homo sapiens
aacttcctaccctttgttaaytggaaaataatcagctctca

SEQ ID NO: 3 - DNA, 41 bp, Homo sapiens
ccagacctattgtgattgttrgtgttactgaaggtctgcag

SEQ ID NO: 4 - DNA, 41 bp, Homo sapiens
ctgcagaccttcagtaacacyaacaatcacaataggtctgg

SEQ ID NO: 5 - DNA, 41 bp, Homo sapiens
gagtctttctgatggtgagcrgggtgatgccataacccggg

SEQ ID NO: 6 - DNA, 41 bp, Homo sapiens
cccgggttatggcatcacccygctcaccatcagaaagactc

SEQ ID NO: 7 - DNA, 41 bp, Homo sapiens
agaagaatgtgtatagcttarcctcaaatctcaaaacctcc

SEQ ID NO: 8 - DNA, 41 bp, Homo sapiens
ggaggttttgagatttgaggytaagctatacacattcttct

SEQ ID NO: 9 - DNA, 41 bp, Homo sapiens
aaagggcatggctcaaatgcygcatcaaattctgctgcccc

SEQ ID NO: 10 - DNA, 41 bp, Homo sapiens
ggggcagcagaatttgatgcrgcatttgagccatgcccttt

SEQ ID NO: 11 - DNA, 41 bp, Homo sapiens
ttccatggagaggcaagtttrgtcttattccaggaatgttt

SEQ ID NO: 12 - DNA, 41 bp, Homo sapiens
aaacattcctggaataagacyaaacttgcctctccatggaa

SEQ ID NO: 13 - DNA, 41 bp, Homo sapiens
cctactgccatgctgaaaagkgtcttgtttgggcaataatg

SEQ ID NO: 14 - DNA, 41 bp, Homo sapiens
cattattgcccaaacaagacmcttttcagcatggcagtagg

SEQ ID NO: 15 - DNA, 41 bp, Homo sapiens
gccttccagaagtgacttctrtaactcatttgtaaatgttg

SEQ ID NO: 16 - DNA, 41 bp, Homo sapiens
caacatttacaaatgagttayagaagtcacttctggaaggc

SEQ ID NO: 17 - DNA, 41 bp, Homo sapiens
tttttgttgagaccccctttratagactctaactctacagt

SEQ ID NO: 18 - DNA, 41 bp, Homo sapiens
actgtagagttagagtctatyaaagggggtctcaacaaaaa

SEQ ID NO: 19 - DNA, 41 bp, Homo sapiens
gatcatgcaggcaacaatctytttggagaaataaatggcat

SEQ ID NO: 20 - DNA, 41 bp, Homo sapiens
atgccatttatttctccaaaragattgttgcctgcatgatc

SEQ ID NO: 21 - DNA, 41 bp, Homo sapiens
tcccatggaaatctgggaagyatgtttctggggcaagtacc

SEQ ID NO: 22 - DNA, 41 bp, Homo sapiens
ggtacttgccccagaaacatrcttcccagatttccatggga

SEQ ID NO: 23 - DNA, 41 bp, Homo sapiens
aggaatgccttcaaaatgagmtgtggatttggggaggttaa

SEQ ID NO: 24 - DNA, 41 bp, Homo sapiens
ttaacctccccaaatccacakctcattttgaaggcattcct

SEQ ID NO: 25 - DNA, 41 bp, Homo sapiens
gggacccattaagagtctatygaaacaaaaaatgcatgaac

SEQ ID NO: 26 - DNA, 41 bp, Homo sapiens
gttcatgcattttttgtttcratagactcttaatgggtccc

SEQ ID NO: 27 - DNA, 41 bp, Homo sapiens
ttctcaagcctattcaaaackcctcccttctcctttgacta

SEQ ID NO: 28 - DNA, 41 bp, Homo sapiens
tagtcaaaggagaagggaggmgttttgaataggcttgagaa

SEQ ID NO: 29 - DNA, 41 bp, Homo sapiens
tccataaaatctgtattctargtcatgtgtagccactgaat

SEQ ID NO: 30 - DNA, 41 bp, Homo sapiens
attcagtggctacacatgacytagaatacagattttatgga

SEQ ID NO: 31 - DNA, 41 bp, Homo sapiens
cttgtaaagattttactcccrtctcacatttatgggaagtt

SEQ ID NO: 32 - DNA, 41 bp, Homo sapiens
aacttcccataaatgtgagaygggagtaaaatctttacaag

SEQ ID NO: 33 - DNA, 41 bp, Homo sapiens
tgttaccatctccaggccctsatgcaatcttttctggcccc

SEQ ID NO: 34 - DNA, 41 bp, Homo sapiens
ggggccagaaaagattgcatsagggcctggagatggtaaca

SEQ ID NO: 35 - DNA, 41 bp, Homo sapiens
acgggaagctggagacctgasgtcatctaggcccctgacac

SEQ ID NO: 36 - DNA, 41 bp, Homo sapiens
gtgtcaggggcctagatgacstcaggtctccagcttcccgt

(19) United States
(12) Patent Application Publication
(10) Pub. No.: US 2012/0141994 A1
(43) Pub. Date: Jun. 7, 2012

(54) METHODS AND COMPOSITIONS FOR PROGNOSING AND DETECTING AGE-RELATED MACULAR DEGENERATION

(76) Inventors: Margaret M. DeAngelis, Bountiful, UT (US); Margaux Morrison, Boston, MA (US)

(21) Appl. No.: 13/318,021

(22) PCT Filed: Apr. 29, 2010
(86) PCT No.: PCT/US10/33052

§ 371 Date: Feb. 21, 2012

Related U.S. Application Data
(60) Provisional application No. 61/173,683, filed on Apr. 29, 2009.

(51) Int. Cl. C12Q 1/68; C07H 21/00; G01N 33/53

(52) U.S. Cl. 435/6.11; 436/501; 536/24.33; 536/24.31

(57) ABSTRACT

The invention provides methods and compositions for determining whether a subject is at risk of developing age-related macular degeneration, for example, the wet or neovascular form of age-related macular degeneration. The method involves determining whether the subject has a protective variant and/or a risk variant at a polymorphic site in the RORA gene. A protective or risk variant may be defined by a haplotype in the RORA gene.

### CROSS-REFERENCE TO RELATED APPLICATIONS

This application claims the benefit of and priority to 61/173,683, filed Apr. 29, 2009; which is incorporated by reference herein in its entirety.

### GOVERNMENT FUNDING

The work described in this application was sponsored, in part, by the National Eye Institute under Grant No. EY014458, EY14104, and EY017362. The United States Government has certain rights in the invention.

### FIELD OF THE INVENTION

The invention relates generally to methods and compositions for determining whether an individual is at risk of developing age-related macular degeneration by detecting whether the individual has a protective or risk variant of the RORA gene.

### BACKGROUND

There are a variety of chronic intraocular disorders, which, if untreated, may lead to partial or even complete vision loss. One prominent chronic intraocular disorder is age-related macular degeneration, which is the leading cause of blindness amongst elderly Americans affecting a third of patients aged 75 years and older (Fine et al. (2000) N. ENGL. J. MED. 342: 483-492). There are two forms of age-related macular degeneration (“AMD”), a dry form and a wet (also known as a neovascular) form.

The dry form involves a gradual degeneration of a specialized tissue beneath the retina, called the retinal pigment epithelium, accompanied by the loss of the overlying photoreceptor cells. These changes result in a gradual loss of vision. The wet form is characterized by the growth of new blood vessels beneath the retina which can bleed and leak fluid, resulting in a rapid, severe and irreversible loss of central vision in the majority cases. This loss of central vision adversely affects one's everyday life by impairing the ability to read, drive and recognize faces. In some cases, the macular degeneration progresses from the dry form to the wet form, and there are at least 200,000 newly diagnosed cases a year of the wet form (Hawkins et al. (1999) MOL. VISION 5: 26-29). The wet form accounts for approximately 90% of the severe vision loss associated with age-related macular degeneration.

At this time, current diagnostic methods cannot accurately predict the risk of age-related macular degeneration for an individual. Unfortunately, the degeneration of the retina has already begun by the time age-related macular degeneration is diagnosed in the clinic. Further, most current treatments are limited in their applicability, and are unable to prevent or reverse the loss of vision especially in the case of the wet type, the more severe form of the disease (Miller et al. (1999) ARCH. OPHTHALMOL. 117(9): 1161-1173).

Currently, the treatment of the dry form of age-related macular degeneration includes administration of antioxidant vitamins and/or zinc. Treatment of the wet form of age-related macular degeneration, however, has proved to be more difficult.

Several methods have been approved in the United States of America for treating the wet form of age-related macular degeneration. Two are laser based approaches, and include laser photocoagulation and photodynamic therapy using a benzoporphyrin derivative photosensitizer known as Visudyne®. Two require the administration of therapeutic molecules that bind and inactivate or reduce the activity of Vascular Endothelial Growth Factor (VEGF), one is known as Lucentis® (ranibizumab), which is a humanized anti-VEGF antibody fragment, and the other is known as Macugen (pegaptanib sodium injection), which is an anti-VEGF aptamer.

During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina creating blind spots with associated vision loss.

During photodynamic therapy, a benzoporphyrin derivative photosensitizer known as Visudyne® and available from QLT, Inc. (Vancouver, Canada) is administered to the individual to be treated. Once the photosensitizer accumulates in the choroidal neovasculature, non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510).

Lucentis®, which is available from Genentech, Inc., CA, is a humanized therapeutic antibody that binds and inhibits or reduces the activity of VEGF, a protein believed to play a role in angiogenesis. Pegaptanib sodium, which is available from OSI Pharmaceuticals, Inc., NY, is a pegylated aptamer that targets VEGF165, the isoform believed to be responsible for primary pathological ocular neovascularization.

The variants and haplotypes most consistently associated with AMD are within the gene complement factor H (CFH) (1q32) and the locus containing the genes age-related maculopathy susceptibility 2 and HtrA serine peptidase 1 (ARMS2 and HTRA1) (10q26) (DeAngelis, et al. (2008) OPHTHALMOL, 115, 1209-1215; Dewan, et al. (2006) SCIENCE, 314, 989-992; Edwards, et al. (2005) SCIENCE, 308, 421-424; Hageman, et al. (2005) PROC. NATL. ACAD. SCI. USA, 102, 7227-7232; Haines, et al. (2005) SCIENCE, 308, 419-421; Jakobsdottir, et al. (2005) AM. J. HUM. GENET., 77, 389-407; Kanda, et al. (2007) PROC. NATL. ACAD. SCI. USA, 104, 16227-16232; Klein, et al. (2005) SCIENCE, 308, 385-389; Li, et al. (2006) NAT. GENET., 38, 1049-1054; Rivera, et al. (2005) HUM. MOL. GENET., 14, 3227-3236; Yang, et al. (2006) SCIENCE, 314, 992-993). These genes have been shown to have large influences on AMD risk in populations of various ethnicities, with variants on 10q26 being the most strongly associated with the neovascular AMD subtype (Fisher, et al. (2005) HUM. MOL. GENET., 14, 2257-2264; Shuler, et al. (2007) ARCH. OPHTHALMOL., 125, 63-67; Zhang, et al. (2008) BMC ED. GENET., 9, 51). Despite their large influence on AMD risk, the combination of these genes alone is insufficient to correctly predict the development and progression of this disease (Jakobsdottir, et al. (2009) PLoS GENET., 5, e1000337).

Therefore, there is still an ongoing need for methods of identifying individuals at risk of developing age-related macular degeneration so that such individuals can be monitored more closely and then treated to slow, stop or reverse the onset of age-related macular degeneration.

### SUMMARY

The invention is based, in part, upon the discovery of single nucleotide polymorphisms (SNPs) and haplotypes located in intron 1 of the retinoic acid-receptor-related orphan receptor α (RORA) gene that are significantly associated with neovascular age-related macular degeneration (AMD) risk. Two single variants at two polymorphic sites have been found to be associated with a reduced risk of developing the neovascular form of AMD as determined by statistical analysis, by virtue of haplotype analysis, and/or by the virtue of the fact that they cluster with variants at polymorphic sites identified by statistical or haplotype analysis. In addition, one haplotype block has been found to be associated with reduced risk of developing the neovascular form of AMD, and a second haplotype block has been found to be associated with increased risk of developing of AMD.

Accordingly, in one aspect, the invention provides a method of determining a subject's, for example, a human subject's, risk of developing age-related macular degeneration.

The method comprises detecting in a sample from a subject the presence or absence of an allelic variant at a polymorphic site of the RORA gene that is associated with risk of developing AMD, such as a protective variant or a risk variant. If the subject has at least one protective variant, the subject is less likely to develop age-related macular degeneration than a person without the protective variant. If the subject has at least one risk variant, the subject is more likely to develop age-related macular degeneration than a person without the risk variant.

In one embodiment, a protective variant G>A (rs4335725) in the RORA gene was identified that is associated with reduced risk of developing the neovascular form of AMD. A person with the protective allele adenine (A) has a lower risk of developing neovascular AMD.

In another embodiment, a protective variant A>G (rs12900948) in the RORA gene was identified that is associated with reduced risk of developing the neovascular form of AMD. A person with the protective allele guanine (G) has a lower risk of developing neovascular AMD.

In another aspect, the invention provides a method of determining a subject's, for example, a human subject's, risk of developing age-related macular degeneration by detecting in a sample from a subject the presence or absence of a haplotype in the RORA gene (or in a region of the RORA gene). If the subject has a protective haplotype, the subject is less likely to develop age-related macular degeneration than a person without the protective haplotype. If the subject has a risk haplotype, the subject is more likely to develop age-related macular degeneration than a person without the risk haplotype.

In one embodiment, a haplotype is defined by the alleles present at the polymorphic sites rs12900948, rs730754, and rs8034864. The method comprises detecting an adenine base or guanine base at rs12900948, an adenine or guanine base at rs730754, and an cytosine base or adenine base at rs8034864. When the haplotype comprises an adenine in the forward sequence of rs12900948, an adenine in the forward sequence of rs730754, and a cytosine in the forward sequence of rs8034864, the haplotype is a risk haplotype indicating that the subject is more likely to develop AMD than a person without this haplotype.

In another embodiment, a haplotype is defined by the alleles present at the polymorphic sites rs17237514 and rs4335725. The method comprises detecting an adenine or guanine base at rs17237514 and an adenine or guanine base at rs4335725. When the haplotype comprises an adenine in the forward sequence of rs17237514 and an adenine in the forward sequence of rs4335725, the haplotype is a protective haplotype indicating that the subject is less likely to develop AMD than a person without this haplotype.

A variant sequence and/or a haplotype can be detected by standard techniques known in the art, which can include, for example, direct nucleotide sequencing, hybridization assays using a probe that anneals to the protective variant, to the risk variant, or to the common allele at the polymorphic site, restriction fragment length polymorphism assays, or amplification-based assays. Furthermore, it is contemplated that the polymorphic sites may be amplified prior to the detection steps. In certain embodiments, the detecting step can include an amplification reaction using primers capable of amplifying the polymorphic site.

In another aspect, the invention provides a method of assisting in diagnosing or assessing the risk of developing age-related macular degeneration. The method can include communicating a report indicating the presence or absence of at least one protective variant at a polymorphic site of the RORA gene in a sample from a subject, for example a human subject. The polymorphic site can include rs12900948 and rs4335725. If the subject has at least one protective variant, the subject is less likely to develop age-related macular degeneration than a person without the protective variant. Alternatively, a protective variant, such as rs12900948 and rs4335725, may be detected by a proxy or surrogate SNP that is in linkage disequilibrium with the protective variant.

In another aspect, the invention provides a method of assisting in diagnosing or assessing the risk of developing age-related macular degeneration. The method can include detecting in a sample from a subject the presence or absence of a haplotype in a region of the RORA gene. If the subject has a risk haplotype, the subject is more likely to develop AMD than a person without the risk haplotype. If the subject has a protective haplotype, the subject is less likely to develop AMD than a person without the protective haplotype. A haplotype may be defined by polymorphic sites (i) rs12900948, rs730754, and rs8034864, and (ii) rs17237514 and rs4335725. Alternatively, a haplotype may be detected by a proxy or surrogate SNP that is in linkage disequilibrium with the haplotype, for example, a haplotype described herein.

The foregoing aspects and embodiments of the invention may be more fully understood by reference to the following detailed description and claims.

### BRIEF DESCRIPTION OF THE DRAWINGS

FIG. 1 provides characteristics of the subjects participating in the studies described herein, including mean age, age range, standard deviation in age, and percentage of male subjects.

FIG. 2 provides the chromosomal location of RORA and the locations of nearby microsatellite markers and SNPs.

FIG. 3A shows results from a Family-Based Association Test (FBAT) using the SNP data gathered from 150 sibling pairs.

FIG. 3B shows results from a conditional logistic regression analysis using the SNP data gathered from 150 sibling pairs.

FIG. 3C shows a stepwise haplotype analysis using the SNP data gathered from 150 sibling pairs. FIG. 3C (i) shows the analysis when rs12916023, rs730754, rs12900948, rs17237514, rs4335725, and rs6494231 are used. FIG. 3C (ii) shows the results of the analysis when rs730754, rs12900948, rs4335725, and rs6494231 are used. FIG. 3C (iii) shows the results of the analysis when rs730754, rs4335725, and rs6494231 are used.

FIG. 4 provides identity-by-state scores from the genotyping results of 18 highly heterozygous microsatellite markers in the 15q region. This analysis identified three markers, D15S1015, D15S209, and D15S214, that were associated with neovascular AMD (p<0.05).

FIG. 5A-B depict linkage disequilibrium plots for the extremely discordant sibling cohort (n=150 sibling pairs). Haplotype blocks were constructed in Haploview using the method proposed by Gabriel et al. (2002) SCIENCE, 296, 2225-2229. Boxes were shaded increasingly darker to represent higher percentage of LD and the numbers listed in each square represent the D′ (FIG. 5A) and r² values (FIG. 5B) unless the box is completely shaded in, representing complete LD. Two haplotype blocks were generated for this cohort.

FIG. 6 shows the family based association test (FBAT) and conditional logistic regression haplotype analysis of RORA in the extremely discordant sibling pair cohort (n=150 sibling pairs). h1 represents haplotype 1; h2 represents haplotype 2; h3 represents haplotype 3. In FIG. 6A, estimated haplotypes with allele frequency greater than 0.05 were listed and tested for association. When considering all possible haplotypes together, the resulting p value from 15,606 permutations was 0.0309. In FIG. 6B, estimated haplotypes with allele frequency greater than 0.05 were listed and tested for association. When considering all possible haplotypes together, the resulting p value from 32,189 permutations was 0.0212. In FIG. 6C, estimated haplotypes with allele frequency greater than 0.05 were listed and tested for association. When considering all possible haplotypes together, the resulting p value from 100,000 permutations was 0.0018.

FIG. 7A-C provide genotype and allele frequencies of the RORA SNPs rs730754, rs8034864, rs12900948, rs17237514 and rs4335725 in the extremely discordant sibling pair cohort (FIG. 7A), discordant sibling pair cohort (FIG. 7B), and Central Greece Cohort (FIG. 7C).

FIG. 8 provides an FBAT analysis of the RORA SNPs rs730754, rs8034864, rs12900948, rs17237514 and rs4335725 in the AREDS category 2 discordant sibling pair cohort (n=46 sibling pairs). One SNP, rs12900948, was associated with decreased risk of developing neovascular AMD under a recessive genetic model (p=0.034).

FIG. 9 depicts linkage disequilibrium plots for the AREDS 2 discordant sibling cohort (n=46 sibling pairs). Haplotype blocks were constructed in Haploview using the method proposed by Gabriel et al., supra. Boxes were shaded increasingly darker to represent higher percentage of LD and the numbers listed in each square represent the D′ (FIG. 9A) and r² values (FIG. 9B) unless the box is completely shaded in representing complete LD. A single haplotype block was generated for this cohort.

FIG. 10 provides haplotype analyses of RORA SNPs in the AREDS category 2 discordant sibling pair cohort (n=46 sibling pairs). FBAT analysis of the two depicted haplotype blocks in the AREDS category 2 discordant sibling pair cohort demonstrated that one haplotype (ACA) within this block was associated with neovascular AMD risk (p=0.0492). However, after permutation testing this finding was no longer significant (p=0.114).

FIG. 11 provides unconditional logistic regression (UCLR) analysis of the RORA SNPs rs730754, rs8034864, rs12900948, rs17237514 and rs4335725 in the Greek cohort. This analysis showed that only rs12900948 was significantly associated with neovascular AMD when comparing neovascular AMD patients to normal patients and separately to patients with early and intermediate dry AMD (AREDS categories 2 and 3) under either a dominant or recessive model. Specifically, the G allele (which is the minor allele for both family-based cohorts, but the major allele for the Greek cohort) of rs12900948 increased risk of neovascular AMD in the unrelated case-control cohort by 4-fold and 3.8-fold when compared to normal patients and separately to patients with dry AMD, respectively.

FIG. 12A-B depict linkage disequilibrium plots for an unrelated case-control cohort from Central Greece. Haplotype blocks were constructed in Haploview using the method proposed by Gabriel et al. supra. Boxes were shaded increasingly darker to represent higher percentage of LD and the numbers listed in each square represent the D′ (FIG. 12A) and r² values (FIG. 12B) unless the box is completely shaded in representing complete LD. A single haplotype block was generated for this cohort.

FIG. 13 provides haplotype analyses of RORA SNPs in the Greek population. When comparing neovascular patients to unaffected patients or separately, to dry patients, the h2 (GAG) haplotype was significantly associated with AMD risk under a dominant model.

### DETAILED DESCRIPTION

As discussed previously, the invention is based, in part, upon the discovery of protective variants and protective and risk haplotypes of the RORA gene that are significantly associated with neovascular AMD risk. Two protective variants, A>G (rs12900948) and G>A (rs4335725) in the RORA gene, have been found to be associated with reduced risk of developing the neovascular form of AMD as determined by statistical analysis, haplotype analysis, or by virtue of the fact that they cluster with variants at polymorphic sites identified by statistical or haplotype analysis. Individuals with one or more protective alleles have lower risk of developing neovascular AMD when compared to those with the respective common alleles.

In addition, two haplotypes associated with risk of AMD were identified. One haplotype was found to be associated with a reduced risk of developing the neovascular form of AMD. This protective haplotype is defined by the polymorphic sites rs4335725 and rs17237514. A second haplotype was found to be associated with increased risk of developing the neovascular form of AMD. This risk haplotype is defined by the polymorphic sites rs12900948, rs730754, and rs8034864.

Although the polymorphic sites rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570 are known, their associations with the risk of developing neovascular AMD, as determined by statistical analysis, haplotype analysis, or by virtue of the fact that they cluster with variants at polymorphic sites identified by statistical or haplotype analysis, heretofore were not known.

RORA is one of three retinoid-related orphan receptors that compose a distinct family of nuclear receptors (Hubbard et al. (2009) NUCL. ACIDS RES., 37, D690-D697). RORA is understood to be a nuclear receptor involved in the regulation of circadian rhythms, the development of cones, bone morphogenesis, and many pathophysiological processes such as cerebellar ataxia, inflammation, atherosclerosis and angiogenesis. (Chauvet et al. (2004) BIOCHEM. J. 384(1):79-85; Besnard et al. (2002) CIRC. RES. 90, 820-825; Besnard et al. (2001) CIRC. RES. 89, 1209-1215; Boukhtouche et al. (2004) ARTERIOSCLER. THROMB. VASC. BIOL. 24, 637-643; Boukhtouche et al. (2006) J. NEUROCHEM. 96, 1778-1789; Lau et al. (2008) J. BIOL. CHEM. 283, 18411-18421; Zhu et al. (2006) ONCOGENE 25, 2901-2908).

The nucleic acid encoding human RORA gene spans approximately 732 kb in length as reported in the NCBI gene database under gene ID: 6095, gene location accession no. NC_{—}000015.8 (58576755 . . . 59308794, complement). The RORA gene has been reported to generate four splicing transcript variants. The transcript variant 1 comprises eleven exons as reported in the NCBI nucleotide database under accession no. NM_{—}134261; the protein encoded by transcript variant 1 is 523 amino acids in length as reported in the NCBI protein database under accession no. NP_{—}599023. The transcript variant 2 comprises twelve exons as reported in the NCBI nucleotide database under accession no. NM_{—}134260; the protein encoded by transcript variant 2 is 556 amino acids in length as reported in the NCBI protein database under accession no. NP_{—}599022. Transcript variant 3 comprises eleven exons as reported in the NCBI nucleotide database under accession no. NM_{—}002943; the protein encoded by transcript variant 3 is 548 amino acids in length as reported in the NCBI protein database under accession no. NP_{—}002934. Transcript variant 4 comprises ten exons as reported in the NCBI nucleotide database under accession no. NM_{—}134262; the protein encoded by transcript variant 4 is 468 amino acids in length as reported in the NCBI protein database under accession no. NP_{—}599024. It is understood that the RORA gene may have more transcript variants. For example, it has been suggested that the RORA gene may generate at least fifteen transcript variants (see the ECGENE database, available at the web site, genome.ewha.ac.kr/ECgene/, under entry H15C5901).

### I. Definitions

The term “polymorphism” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and may be homozygous or heterozygous for allelic forms.

A “polymorphic ^{site} is the position or locus at which sequence divergence occurs at the nucleic acid level and is sometimes reflected at the amino acid level. The polymorphic region or polymorphic site refers to a region of the nucleic acid where the nucleotide difference that distinguishes the variants occurs, or, for amino acid sequences, a region of the amino acid sequence where the amino acid difference that distinguishes the protein variants occurs. A polymorphic site can be as small as one base pair, often termed a “single nucleotide polymorphism” (SNP). The SNPs can be any SNPs in loci identified herein, including intragenic SNPs in exons, introns, or upstream or downstream regions of a gene, as well as SNPs that are located outside of gene sequences. Examples of such SNPs include, but are not limited to rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570.

The term “genotype” as used herein denotes one or more polymorphisms of interest found in an individual, for example, within a gene of interest. Diploid individuals have a genotype that comprises two different sequences (heterozygous) or one sequence (homozygous) at a polymorphic site.

The term “haplotype” refers to a DNA sequence comprising one or more polymorphisms of interest contained on a subregion of a single chromosome of an individual. A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms on chromosome 15 near the RORA gene, e.g. within the gene and/or within intergenic sequences (i.e., intervening intergenic sequences, upstream sequences, and downstream sequences that are in linkage disequilibrium with polymorphisms in the genic region). The term “haplotype” can refer to a set of single nucleotide polymorphisms (SNPs) found to be statistically associated on a single chromosome. A haplotype can also refer to a combination of polymorphisms (e.g., SNPs) and other genetic markers found to be statistically associated on a single chromosome. A haplotype, for instance, can also be a set of maternally inherited alleles, or a set of paternally inherited alleles, at any locus.

The term “genetic profile,” as used herein, refers to a collection of one or more polymorphic sites including rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570, optionally in combination with other genetic characteristics such as deletions, additions or duplications, and optionally combined with other polymorphic sites associated with AMD risk or protection. Thus, a genetic profile, as the phrase is used herein, is not limited to a set of characteristics defining a haplotype, and may include polymorphic sites from diverse regions of the genome. For example, a genetic profile for AMD includes one or a subset of single nucleotide polymorphisms such as rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570, optionally in combination with other genetic characteristics associated with AMD. It is understood that while one polymorphic site in a genetic profile may be informative of an individual's increased or decreased risk (i.e., an individual's propensity or susceptibility) to develop AMD, more than one polymorphic site in a genetic profile may and typically will be analyzed and will be more informative of an individual's increased or decreased risk of developing AMD. A genetic profile may include at least one SNP disclosed herein in combination with other polymorphisms or genetic markers and/or environmental factors (e.g., smoking or obesity) known to be associated with AMD. In some cases, a polymorphic site may reflect a change in regulatory or protein coding sequences that change gene product levels or activity in a manner that results in increased likelihood of development of disease. In addition, it will be understood by a person of skill in the art that one or more polymorphic sites that are part of a genetic profile may be in linkage disequilibrium with, and serve as a proxy or surrogate marker for, another genetic marker or polymorphism that is causative, protective, or otherwise informative of disease.

The term “gene,” as used herein, refers to a region of a DNA sequence that encodes a polypeptide or protein, intronic sequences, promoter regions, and upstream (i.e., proximal) and downstream (i.e., distal) non-coding transcription control regions (e.g., enhancer and/or repressor regions).

The term “allele,” as used herein, refers to a sequence variant of a genetic sequence (e.g., typically a gene sequence as described hereinabove, optionally a protein coding sequence). For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs. In certain embodiments of the invention, an allele is defined by the genotypes of at least 1, 2, 4, 8 or 16 or more SNPs, (including, but not limited to, rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and/or rs11071570) in a gene.

A “causative” polymorphic site is a polymorphic site (e.g., a SNP) having an allele that is directly responsible for a difference in risk of development or progression of AMD. Generally, a causative polymorphic site has an allele producing an alteration in gene expression or in the expression, structure, and/or function of a gene product, and therefore is most predictive of a possible clinical phenotype. One such class includes polymorphic sites falling within regions of genes encoding a polypeptide product, i.e. “coding polymorphic sites” (e.g., “coding SNPs” (cSNPs)). These polymorphic sites may result in an alteration of the amino acid sequence of the polypeptide product (i.e., non-synonymous codon changes) and give rise to the expression of a defective or other variant protein. Furthermore, in the case of nonsense mutations, a polymorphic site may lead to premature termination of a polypeptide product. Such variant products can result in a pathological condition, e.g., genetic disease. Examples of genes in which a polymorphic site within a coding sequence causes a genetic disease include sickle cell anemia and cystic fibrosis.

Causative polymorphic sites do not necessarily have to occur in coding regions; causative polymorphic sites can occur in, for example, any genetic region that can ultimately affect the expression, structure, and/or activity of the protein encoded by a nucleic acid. Such genetic regions include, for example, those involved in transcription, such as polymorphic sites in transcription factor binding domains, polymorphic sites in promoter regions, in areas involved in transcript processing, such as polymorphic sites at intron-exon boundaries that may cause defective splicing, or polymorphic sites in mRNA processing signal sequences such as polyadenylation signal regions. Some polymorphic sites that are not causative polymorphic sites nevertheless are in close association with, and therefore segregate with, a disease-causing sequence. In this situation, the presence of an allele at the polymorphic site correlates with the presence of, or predisposition to, or an increased risk in developing the disease. These polymorphic sites, although not causative, are nonetheless also useful for diagnostics, disease predisposition screening, and other uses.

The term “linkage” refers to the tendency of genes, alleles, loci, or genetic markers to be inherited together as a result of their location on the same chromosome or as a result of other factors. Linkage can be measured by percent recombination between the two genes, alleles, loci, or genetic markers. Some linked markers may be present within the same gene or gene cluster.

In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium is influenced by a number of factors including genetic linkage, the rate of recombination, the rate of mutation, random drift, non-random mating, and population structure. “Linkage disequilibrium” or “allelic association” thus means the preferential association of a particular allele or genetic marker with another specific allele or genetic marker more frequently than expected by chance for any particular allele frequency in the population. A marker in linkage disequilibrium with a risk or protective variant, such as those at rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570, can be useful in detecting susceptibility to disease. A polymorphic variant that is in linkage disequilibrium with a causative, risk-associated, protective, or otherwise informative polymorphic variant or genetic marker is referred to as a “proxy” or “surrogate” polymorphic variant. A proxy polymorphic variant may be in at least 50%, 60%, or 70% in linkage disequilibrium with the causative polymorphic variant, and preferably is at least about 80%, 90%, and most preferably 95%, or about 100% in LD with the genetic marker.

A “nucleic acid,” “polynucleotide,” or “oligonucleotide” is a polymeric form of nucleotides of any length, may be DNA or RNA, and may be single- or double-stranded. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA) and any other polymers capable of serving as a template for an amplification reaction using an amplification technique, for example, a polymerase chain reaction, a ligase chain reaction, or non-enzymatic template-directed replication.

“Hybridization probes” are nucleic acids capable of binding in a base-specific manner to a complementary strand of nucleic acid. Such probes include nucleic acids and peptide nucleic acids. Hybridization is usually performed under stringent conditions which are known in the art. A hybridization probe may include a “primer.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The nucleic acids, including any primers, probes and/or oligonucleotides can be synthesized using a variety of techniques currently available, such as by chemical or biochemical synthesis, and by in vitro or in vivo expression from recombinant nucleic acid molecules, e.g., bacterial or retroviral vectors. For example, DNA can be synthesized using conventional nucleotide phosphoramidite chemistry and the instruments available from Applied Biosystems, Inc. (Foster City, Calif.); DuPont (Wilmington, Del.); or Milligen (Bedford, Mass.). When desired, the nucleic acids can be labeled using methodologies well known in the art such as described in U.S. Pat. Nos. 5,464,746; 5,424,414; and 4,948,882 all of which are herein incorporated by reference. In addition, the nucleic acids can comprise uncommon and/or modified nucleotide residues or non-nucleotide residues, such as those known in the art.

“Stringent” as used herein refers to hybridization and wash conditions at 50° C. or higher. Other stringent hybridization conditions may also be selected. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 50° C. As other factors may significantly affect the stringency of hybridization, including, among others, base composition, length of the nucleic acid strands, the presence of organic solvents, and the extent of base mismatching, the combination of parameters is more important than the absolute measure of any one.

The terms “susceptibility” and “risk” refer to either an increased or decreased likelihood of an individual developing a disorder (e.g., a condition, illness, disorder or disease) relative to a control and/or non-diseased population or to progressing from one form of a disorder to another relative to a control and/or a population having the initial form of the disorder. In one example, the control population may be individuals in the population (e.g., matched by age, gender, race and/or ethnicity) without the disorder, or without the genotype or phenotype assayed for. In another example, the control population may be individuals with the dry form of AMD (e.g., matched by age, gender, race and/or ethnicity), such as when considering risk of progressing from the dry form of AMD to the wet form of AMD.

The terms “diagnose” and “diagnosis” refer to the ability to determine or identify whether an individual has a particular disorder (e.g., a condition, illness, disorder or disease). The term “prognose” or “prognosis” refers to the ability to predict the course of the disease (including to predict the risk of developing the disease) and/or to predict the likely outcome of a particular therapeutic or prophylactic strategy.

The term “screen” or “screening” as used herein has a broad meaning. It includes processes intended for diagnosing or for determining the susceptibility, propensity, risk, or risk assessment of an asymptomatic subject for developing a disorder later in life. Screening also includes the prognosis of a subject, i.e., when a subject has been diagnosed with a disorder, determining in advance the progress of the disorder as well as the assessment of efficacy of therapy options to treat a disorder. Screening can be done by examining a presenting individual's DNA, RNA, or in some cases, protein, to assess the presence or absence of the various polymorphic variants disclosed herein (and typically other polymorphic variants and genetic or behavioral characteristics) so as to determine where the individual lies on the spectrum of disease risk-neutrality-protection. Proxy polymorphic variants may substitute for any of these polymorphic variants. A sample such as a blood sample may be taken from the individual for purposes of conducting the genetic testing using methods known in the art or yet to be developed. Alternatively, if a health provider has access to a pre-produced data set recording all or part of the individual's genome (e.g. a listing of polymorphic variants in the individual's genome), screening may be done simply by inspection of the database, optimally by computerized inspection. Screening may further comprise the step of producing a report identifying the individual and the identity of alleles at the site of at least one or more of the rs730754, rs975501, rs12900948, rs782925, rs7177611, rs16943429, rs2414687, rs4335725, rs6494231, rs12916023, rs4583176, rs8034864, rs1403737, rs12591914, rs7495128, rs17237514, rs17270640, and rs11071570 SNPs.

### II. Prognosis and Diagnosis of Neovascular AMD

In one aspect, the invention provides a method of determining a subject's, for example, a human subject's, risk of developing age-related macular degeneration. The method comprises detecting in a sample, for example, a tissue, body fluid, or cell-containing sample, from a subject the presence or absence of an allelic variant at a polymorphic site of the RORA gene that is associated with risk of developing AMD, such as a protective variant or a risk variant. In an exemplary embodiment, the method comprises determining whether the subject has a protective variant at a polymorphic site of the RORA gene, wherein, if the subject has at least one protective variant, the subject is less likely to develop age-related macular degeneration than a person without the protective variant. Two exemplary protective variants are located in intron 1 of the RORA gene.

In one exemplary embodiment, a protective variant G>A (rs4335725) in the RORA gene was identified that is associated with reduced risk of developing the neovascular form of AMD. A person with the protective allele A has a lower risk of developing neovascular AMD. Throughout the specification, protective and risk variants are referred to using the following exemplary designation “G>A (rs4335725).” Using this convention, the first nucleotide base refers to the common allele (also referred to as the major allele) followed the “>” symbol then the variant allele (also referred to as the minor allele or rare allele). In some instances, the polymorphic site designation is provided in parentheses.

In another exemplary embodiment, a protective variant A>G (rs12900948) in the RORA gene was identified that is associated with reduced risk of developing the neovascular form of AMD. A person with the protective allele G has a lower risk of developing neovascular AMD.

In other embodiments, protective variants may be determined at the following polymorphic sites: rs730754, rs975501, rs782925, rs7177611, rs16943429, rs2414687, and rs6494231 in the RORA gene, as described herein.

In one embodiment, a protective variant A>G (rs730754) in the RORA gene was identified that is associated with reduced risk of the neovascular form of AMD. A person with the protective allele G has a lower risk of developing neovascular AMD.

In another embodiment, a protective variant A>G (rs975501) in the RORA gene was identified that is associated with reduced risk of developing the neovascular form of AMD. A person with the protective allele G has a lower risk of developing neovascular AMD.

In another embodiment, a protective variant G>A (rs782925) in the RORA gene was identified that is associated with reduced risk of developing the neovascular form of AMD. A person with the protective allele A has a lower risk of developing neovascular AMD.

In another embodiment, a protective variant C>T (rs7177611) in the RORA gene was identified that is associated with reduced risk of developing the neovascular form of AMD. A person with the protective allele T has a lower risk of developing neovascular AMD.

In another embodiment, a protective variant A>G (rs16943429) in the RORA gene was identified that is associated with reduced risk of developing the neovascular form of AMD. A person with the protective allele G has a lower risk of developing neovascular AMD.

In another embodiment, a protective variant G>T (rs2414687) in the RORA gene was identified that is associated with reduced risk of developing the neovascular form of AMD. A person with the protective allele T has a lower risk of developing neovascular AMD.

In another embodiment, a protective variant G>A (rs6494231) in the RORA gene was identified that is associated with reduced risk of developing the neovascular form of AMD. A person with the protective allele A has a lower risk of developing neovascular AMD.

An exemplary protective variant is at a SNP, rs4335725 located in intron 1 of the RORA gene. For example, the forward sequence comprises GCCTTCCAGAAGTGACTTCT[X₁₅]TAACTCATTTGTAAATGTTG (SEQ ID NO. 15) wherein X₁₅ is a G to an A substitution. G is the common allele, and A is the protective variant. Alternatively, the reverse sequence comprises CAACATTTACAAATGAGTTA[X₁₆]AGAAGTCACTTCTGGAAGGC (SEQ ID NO. 16) wherein X₁₆ is a C to a T substitution. C is the common allele, and T is the protective variant. rs4335725 is a single nucleotide polymorphism with a G to an A substitution in the forward sequence or a C to a T substitution in the reverse sequence at chromosome 15 base pair position 59027896.

Another exemplary protective variant is at a SNP, rs12900948 located in intron 1 of the RORA gene. For example, the forward sequence comprises GAGTCTTTCTGATGGTGAGC[X₅]GGGTGATGCCATAACCCGGG (SEQ ID NO. 5) wherein X₅ is an A to a G substitution. A is the common allele, and G is the protective variant. Alternatively, the reverse sequence comprises CCCGGGTTATGGCATCACCC[X₆]GCTCACCATCAGAAAGACTC (SEQ ID NO. 6) wherein X₆ is a T to a C substitution. T is the common allele, and C is the protective variant. rs12900948 is a single nucleotide polymorphism with an A to a G substitution in the forward sequence or a T to a C substitution in the reverse sequence at chromosome 15 base pair position 59227963.

Other protective variants are disclosed below. Another protective variant is at a SNP, rs730754, located in intron 1 of the RORA gene. For example, the forward sequence comprises TGAGAGCTGATTATTTTCCA[X₁]TTAACAAAGGGTAGGAAGTT (SEQ ID NO. 1) wherein X₁ is an A to a G substitution. A is the common allele, and G is the protective variant. Alternatively, the reverse sequence comprises AACTTCCTACCCTTTGTTAA[X₂]TGGAAAATAATCAGCTCTCA (SEQ ID NO. 2) wherein X₂ is a T to a C substitution. T is the common allele, and C is the protective variant. rs730754 is a single nucleotide polymorphism with an A to a G substitution in the forward sequence or a T to a C substitution in the reverse sequence at chromosome 15 base pair position 59238628.

Another protective variant is at a SNP, rs975501 located in intron 1 of the RORA gene. For example, the forward sequence comprises CCAGACCTATTGTGATTGTT[X₃]GTGTTACTGAAGGTCTGCAG (SEQ ID NO. 3) wherein X₃ is an A to a G substitution. A is the common allele, and G is the protective variant. Alternatively, the reverse sequence comprises CTGCAGACCTTCAGTAACAC[X₄]AACAATCACAATAGGTCTGG (SEQ ID NO. 4) wherein X₄ is a T to a C substitution. T is the common allele, and C is the protective variant. rs975501 is a single nucleotide polymorphism with an A to a G substitution in the forward sequence or a T to a C substitution in the reverse sequence at chromosome 15 base pair position 59228713.

Another protective variant is at a SNP, rs782925 located in intron 1 of the RORA gene. For example, the forward sequence comprises AGAAGAATGTGTATAGCTTA[₇]CCTCAAATCTCAAAACCTCC (SEQ ID NO. 7) wherein X₇ is a G to an A substitution. G is the common allele, and A is the protective variant. Alternatively, the reverse sequence comprises GGAGGTTTTGAGATTTGAGG[X₈]TAAGCTATACACATTCTTCT (SEQ ID NO. 8) wherein X₈ is a C to a T substitution. C is the common allele, and T is the protective variant. rs782925 is a single nucleotide polymorphism with a G to an A substitution in the forward sequence or a C to a T substitution in the reverse sequence at chromosome 15 base pair position 59190716.

Another protective variant is at a SNP, rs7177611 located in intron 1 of the RORA gene. For example, the forward sequence comprises AAAGGGCATGGCTCAAATGC[X₉]GCATCAAATTCTGCTGCCCC (SEQ ID NO. 9) wherein X₉ is a C to a T substitution. C is the common allele, and T is the protective variant. Alternatively, the reverse sequence comprises GGGGCAGCAGAATTTGATGC[X₁₀]GCATTTGAGCCATGCCCTTT (SEQ ID NO. 10) wherein X₁₀ is an G to an A substitution. G is the common allele, and A is the protective variant. rs7177611 is a single nucleotide polymorphism with a C to a T substitution in the forward sequence or a G to an A substitution in the reverse sequence at chromosome 15 base pair position 59160051.

Another protective variant is at a SNP, rs16943429 located in intron 1 of the RORA gene. For example, the forward sequence comprises TTCCATGGAGAGGCAAGTTT[X₁₁]GTCTTATTCCAGGAATGTTT (SEQ ID NO. 11) wherein X₁₁ is an A to a G substitution. A is the common allele, and G is the protective variant. Alternatively, the reverse sequence comprises AAACATTCCTGGAATAAGAC[X₁₂]AAACTTGCCTCTCCATGGAA (SEQ ID NO. 12) wherein X₁₂ is a T to a C substitution. T is the common allele, and C is the protective variant. rs16943429 is a single nucleotide polymorphism with an A to a G substitution in the forward sequence or a T to a C substitution in the reverse sequence at chromosome 15 base pair position 59066545.

Another protective variant is at a SNP, rs2414687 located in intron 1 of the RORA gene. For example, the forward sequence comprises CCTACTGCCATGCTGAAAAG[X₁₃]GTCTTGTTTGGGCAATAATG (SEQ ID NO. 13) wherein X₁₃ is a G to a T substitution. G is the common allele, and T is the protective variant. Alternatively, the reverse sequence comprises CATTATTGCCCAAACAAGAC[X₁₄]CTTTTCAGCATGGCAGTAGG (SEQ ID NO. 14) wherein X₁₄ is an C to an A substitution. C is the common allele, and A is the protective variant. rs2414687 is a single nucleotide polymorphism with a G to a T substitution in the forward sequence or a C to an A substitution in the reverse sequence at chromosome 15 base pair position 59056537.

Another protective variant is at a SNP, rs6494231 located in intron 1 of the RORA gene. For example, the forward sequence comprises TTTTTGTTGAGACCCCCTTT[X₁₇]ATAGACTCTAACTCTACAGT (SEQ ID NO. 17) wherein X₁₇ is a G to an A substitution. G is the common allele, and A is the protective variant. Alternatively, the reverse sequence comprises ACTGTAGAGTTAGAGTCTAT[X₁₈]AAAGGGGGTCTCAACAAAAA (SEQ ID NO. 18) wherein X₁₈ is a C to a T substitution. C is the common allele, and T is the protective variant. rs6494231 is a single nucleotide polymorphism with a G to an A substitution in the forward sequence or a C to a T substitution in the reverse sequence at chromosome 15 base pair position 58993284.

In another aspect, the invention provides a method of determining a subject's, for example, a human subject's, risk of developing age-related macular degeneration. The method comprises determining whether the subject has a risk variant at a polymorphic site of the RORA gene, wherein, if the subject has at least one risk variant, the subject is more likely to develop age-related macular degeneration than a person without the risk variant. Risk variants located in intron 1 of the RORA gene may include rs12916023, rs4583176, rs8034864, rs1403737, rs12591914, rs7495128, rs17237514, rs17270640, and rs11071570.

In one embodiment, a risk variant T>C (rs12916023) in the RORA gene was identified that is associated with increased risk of developing the neovascular form of AMD. A person with the risk allele C has a higher risk of developing neovascular AMD.

In another embodiment, a risk variant C>T (rs4583176) in the RORA gene was identified that is associated with increased risk of developing the neovascular form of AMD. A person with the risk allele T has a higher risk of developing neovascular AMD.

In another embodiment, a risk variant C>A (rs8034864) in the RORA gene was identified that is associated with increased risk of developing the neovascular form of AMD. A person with the risk allele A has a higher risk of developing neovascular AMD.

In another embodiment, a risk variant C>T (rs1403737) in the RORA gene was identified that is associated with increased risk of developing the neovascular form of AMD. A person with the risk allele T has a higher risk of developing neovascular AMD.

In another embodiment, a risk variant G>T (rs12591914) in the RORA gene was identified that is associated with increased risk of developing the neovascular form of AMD. A person with the risk allele T has a higher risk of developing neovascular AMD.

In another embodiment, a risk variant G>A (rs7495128) in the RORA gene was identified that is associated with increased risk of developing the neovascular form of AMD. A person with the risk allele A has a higher risk of developing neovascular AMD.

In another embodiment, a risk variant A>G (rs17237514) in the RORA gene was identified that is associated with increased risk of developing the neovascular form of AMD. A person with the risk allele G has a higher risk of developing neovascular AMD.

In another embodiment, a risk variant C>G (rs17270640) in the RORA gene was identified that is associated with increased risk of developing the neovascular form of AMD. A person with the risk allele G has a higher risk of developing neovascular AMD.

In another embodiment, a risk variant G>C (rs11071570) in the RORA gene was identified that is associated with increased risk of developing the neovascular form of AMD. A person with the risk allele C has a higher risk of developing neovascular AMD.

One risk variant is at a SNP, rs12916023 located in intron 1 of the RORA gene. For example, the forward sequence comprises GATCATGCAGGCAACAATCT[X₁₉]TTTGGAGAAATAAATGGCAT (SEQ ID NO. 19) wherein X₁₉ is a T to a C substitution. T is the common allele, and C is the risk variant. Alternatively, the reverse sequence comprises ATGCCATTTATTTCTCCAAA[X₂₀]AGATTGTTGCCTGCATGATC (SEQ ID NO. 20) wherein X₂₀ is an A to a G substitution. A is the common allele, and G is the risk variant. rs12916023 is a single nucleotide polymorphism with a T to a C substitution in the forward sequence or an A to a G substitution in the reverse sequence at chromosome 15 base pair position 59243345.

Another risk variant is at a SNP, rs4583176 located in intron 1 of the RORA gene. For example, the forward sequence comprises TCCCATGGAAATCTGGGAAG[X₂₁]ATGTTTCTGGGGCAAGTACC (SEQ ID NO. 21) wherein X₂₁ is a C to a T substitution. C is the common allele, and T is the risk variant. Alternatively, the reverse sequence comprises GGTACTTGCCCCAGAAACAT[X₂₂]CTTCCCAGATTTCCATGGGA (SEQ ID NO. 22) wherein X₂₂ is an G to an A substitution. G is the common allele, and A is the risk variant.

rs4583176 is a single nucleotide polymorphism with a C to a T substitution in the forward sequence or a G to an A substitution in the reverse sequence at chromosome 15 base pair position 59240403.

Another risk variant is at a SNP, rs8034864 located in intron 1 of the RORA gene. For example, the forward sequence comprises AGGAATGCCTTCAAAATGAG[X₂₃]TGTGGATTTGGGGAGGTTAA (SEQ ID NO. 23) wherein X₂₃ is a C to an A substitution. C is the common allele, and A is the risk variant. Alternatively, the reverse sequence comprises TTAACCTCCCCAAATCCACA[X₂₄]CTCATTTTGAAGGCATTCCT (SEQ ID NO. 24) wherein X₂₄ is a G to a T substitution. G is the common allele, and T is the risk variant. rs8034864 is a single nucleotide polymorphism with a C to an A substitution in the forward sequence or a G to a T substitution in the reverse sequence at chromosome 15 base pair position 59233645.

Another risk variant is at a SNP, rs1403737 located in intron 1 of the RORA gene. For example, the forward sequence comprises GGGACCCATTAAGAGTCTAT[X₂₅]GAAACAAAAAATGCATGAAC (SEQ ID NO. 25) wherein X₂₅ is a C to a T substitution. C is the common allele, and T is the risk variant. Alternatively, the reverse sequence comprises GTTCATGCATTTTTTGTTTC[X₂₆]ATAGACTCTTAATGGGTCCC (SEQ ID NO. 26) wherein X₂₆ is an G to an A substitution. G is the common allele, and A is the risk variant. rs1403737 is a single nucleotide polymorphism with a C to a T substitution in the forward sequence or a G to an A substitution in the reverse sequence at chromosome 15 base pair position 59103494.

Another risk variant is at a SNP, rs12591914 located in intron 1 of the RORA gene. For example, the forward sequence comprises TTCTCAAGCCTATTCAAAAC[X₂₇]CCTCCCTTCTCCTTTGACTA (SEQ ID NO. 27) wherein X₂₇ is a G to a T substitution. G is the common allele, and T is the risk variant. Alternatively, the reverse sequence comprises TAGTCAAAGGAGAAGGGAGG[X₂₈]GTTTTGAATAGGCTTGAGAA (SEQ ID NO. 28) wherein X₂₈ is an C to an A substitution. C is the common allele, and A is the risk variant. rs12591914 is a single nucleotide polymorphism with a G to a T substitution in the forward sequence or a C to an A substitution in the reverse sequence at chromosome 15 base pair position 59073949.

Another risk variant is at a SNP, rs7495128 located in intron 1 of the RORA gene. For example, the forward sequence comprises TCCATAAAATCTGTATTCTA[X₂₉]GTCATGTGTAGCCACTGAAT (SEQ ID NO. 29) wherein X₂₉ is a G to an A substitution. G is the common allele, and A is the risk variant. Alternatively, the reverse sequence comprises ATTCAGTGGCTACACATGAC[X₃₀]TAGAATACAGATTTTATGGA (SEQ ID NO. 30) wherein X₃₀ is a C to a T substitution. C is the common allele, and T is the risk variant. rs7495128 is a single nucleotide polymorphism with a G to an A substitution in the forward sequence or a C to a T substitution in the reverse sequence at chromosome 15 base pair position 59061522.

Another risk variant is at a SNP, rs17237514 located in intron 1 of the RORA gene. For example, the forward sequence comprises CTTGTAAAGATTTTACTCCC[X₃₁]TCTCACATTTATGGGAAGTT (SEQ ID NO. 31) wherein X₃₁ is an A to a G substitution. A is the common allele, and G is the risk variant. Alternatively, the reverse sequence comprises AACTTCCCATAAATGTGAGA[X₃₂]GGGAGTAAAATCTTTACAAG (SEQ ID NO. 32) wherein X₃₂ is a T to a C substitution. T is the common allele, and C is the risk variant. rs17237514 is a single nucleotide polymorphism with an A to a G substitution in the forward sequence or a T to a C substitution in the reverse sequence at chromosome 15 base pair position 59033967.

Another risk variant is at a SNP, rs17270640 located in intron 1 of the RORA gene. For example, the forward sequence comprises TGTTACCATCTCCAGGCCCT[X₃₃]ATGCAATCTTTTCTGGCCCC (SEQ ID NO. 33) wherein X₃₃ is a C to a G substitution. C is the common allele, and G is the risk variant. Alternatively, the reverse sequence comprises GGGGCCAGAAAAGATTGCAT[X₃₄]AGGGCCTGGAGATGGTAACA (SEQ ID NO. 34) wherein X₃₄ is a G to a C substitution. G is the common allele, and C is the risk variant. rs17270640 is a single nucleotide polymorphism with a C to a G substitution in the forward sequence or a G to a C substitution in the reverse sequence at chromosome 15 base pair position 59005595.

Another risk variant is at a SNP, rs11071570 located in intron 1 of the RORA gene. For example, the forward sequence comprises ACGGGAAGCTGGAGACCTGA[X₃₅]GTCATCTAGGCCCCTGACAC (SEQ ID NO. 35) wherein X₃₅ is a G to a C substitution. G is the common allele, and C is the risk variant. Alternatively, the reverse sequence comprises GTGTCAGGGGCCTAGATGAC[X₃₆]TCAGGTCTCCAGCTTCCCGT (SEQ ID NO. 36) wherein X₃₆ is an C to a G substitution. C is the common allele, and G is the risk variant. rs11071570 is a single nucleotide polymorphism with a G to a C substitution in the forward sequence or a C to a G substitution in the reverse sequence at chromosome 15 base pair position 59002983.

In another aspect, the invention provides a method of determining a subject's, for example, a human subject's, risk of developing age-related macular degeneration. The method comprises detecting in a sample from a subject the presence or absence of a haplotype in the RORA gene. If the subject has a protective haplotype, the subject is less likely to develop age-related macular degeneration than a person without the protective haplotype. If the subject has a risk haplotype, the subject is more likely to develop age-related macular degeneration than a person without the risk haplotype.

In one embodiment, a haplotype is defined by the alleles present at the polymorphic sites rs12900948, rs730754, and rs8034864. The method comprises detecting an adenine or guanine base at rs12900948, an adenine or guanine base at rs730754, and an cytosine or adenine base at rs8034864. When the haplotype comprises an adenine in the forward sequence of rs12900948, an adenine in the forward sequence of rs730754, and a cytosine in the forward sequence of rs8034864, the haplotype is a risk haplotype indicating that the subject is more likely to develop AMD than a person without this haplotype.

In another embodiment, a haplotype is defined by the alleles present at the polymorphic sites rs17237514 and rs4335725. The method comprises detecting an adenine or guanine base at rs17237514 and an adenine or guanine base at rs4335725. When the haplotype comprises an adenine in the forward sequence of rs17237514 and an adenine in the forward sequence of rs4335725, the haplotype is a protective haplotype indicating that the subject is less likely to develop AMD than a person without this haplotype.

In some embodiments, a protective variant and/or a risk variant of the RORA gene, and/or a protective haplotype and/or a risk haplotype of the RORA gene may be detected in combination with a protective variant and/or a risk variant at one or more of the following polymorphic sites: rs1061170 (CFH), rs800292 (CFH), rs10490924 (LOC387715) and rs11200638 (ARMS2/HTRA1).

The presence of a protective and/or risk variant (and/or a protective and/or risk haplotype) can be determined by standard nucleic acid detection assays including, for example, conventional SNP detection assays, which may include, for example, amplification-based assays, probe hybridization assays, restriction fragment length polymorphism assays, and/or direct nucleic acid sequencing. Exemplary protocols for preparing and analyzing samples of interest are discussed in the following sections.

A. Preparation of Samples for Analysis

Polymorphisms can be detected in a target nucleic acid sample from an individual under investigation. In general, genomic DNA can be analyzed, which can be selected from any biological sample that contains genomic DNA or RNA. For example, genomic DNA can be obtained from peripheral blood leukocytes using standard approaches (QIAamp DNA Blood Maxi kit, Qiagen, Valencia, Calif.). Nucleic acids can be harvested from other samples, for example, cells in saliva, cheek scrapings, amniotic fluid, placental tissue, urine, hair, skin, blood, biopsies of the retina, kidney, or liver or other organs or tissues. Methods for purifying nucleic acids from biological samples suitable for use in diagnostic or other assays are known in the art.

Alternatively, an individual's genetic profile may be analyzed by inspecting a data set indicative of genetic characteristics previously derived from analysis of the individual's genome. A data set indicative of an individual's genetic characteristics may include a complete or partial sequence of the individual's genomic DNA, or a SNP map. Inspection of the data set including all or part of the individual's genome may optimally be performed by computer inspection. Screening may further comprise the step of producing a report identifying the individual and the identity of alleles at the site of at least one or more of the rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570 SNPs and/or proxy polymorphic sites.

B. Detection of Polymorphisms in Target Nucleic Acids

The identity of bases present at the polymorphic sites, rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570, and/or rs11071570, can be determined in an individual using any of several methods known in the art. The polymorphisms can be detected by direct sequencing, amplification-based assays, probe hybridization-based assays, restriction fragment length polymorphism assays, denaturing gradient gel electrophoresis, single-strand conformation polymorphism analyses, and denaturing high performance liquid chromatography. Other methods to detect nucleic acid polymorphisms include the use of: Molecular Beacons (see, e.g., Piatek et al. (1998) NAT. BIOTECHNOL. 16:359-63; Tyagi and Kramer (1996) NAT. BIOTECHNOL. 14:303-308; and Tyagi et al. (1998) NAT. BIOTECHNOL. 16:49-53), the Invader assay (see, e.g., Neri et al. (2000) ADV. NUCL. ACID PROTEIN ANALYSIS 3826: 117-125 and U.S. Pat. No. 6,706,471), and the Scorpion assay (Thelwell et al. (2000) NUCL. ACIDS RES. 28:3752-3761 and Solinas et al. (2001) NUCL. ACIDS RES. 29:20).

The design and use of allele-specific probes for analyzing polymorphisms are described, for example, in EP 235,726, and WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes are designed to hybridize to a segment of target DNA such that the polymorphic site aligns with a central position of the probe.

Probe-based genotyping can be carried out using a “TaqMan” or “5′-nuclease assay,” as described in U.S. Pat. Nos. 5,210,015; 5,487,972; and 5,804,375; and Holland et al. (1988) PROC. NATL. ACAD. SCI. USA 88:7276-7280, each incorporated herein by reference. Examples of other techniques that can be used for polymorphic site genotyping include, but are not limited to, Amplifluor, Dye Binding-Intercalation, Fluorescence Resonance Energy Transfer (FRET), Hybridization Signal Amplification Method (HSAM), HYB Probes, Invader/Cleavase Technology (Invader/CFLP), Molecular Beacons, Origen, DNA-Based Ramification Amplification (RAM), rolling circle amplification, Scorpions, Strand displacement amplification (SDA), oligonucleotide ligation (Nickerson et al. (1990) PROC. NATL ACAD. SCI. USA 87:8923-8927) and/or enzymatic cleavage. Popular high-throughput polymorphic variant detection (e.g., SNP variant detection) methods also include template-directed dye-terminator incorporation (TDI) assay (Chen and Kwok (1997) NUCL. ACIDS RES. 25:347-353), the 5′-nuclease allele-specific hybridization TaqMan assay (Livak et al. (1995) NATURE GENET. 9:341-342), and the allele-specific molecular beacon assay (Tyagi et al. (1998) NATURE BIOTECH. 16:49-53).

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference. In some embodiments multiple assays are conducted using a microfluidic format. (See, e.g., Unger et al. (2000) SCIENCE 288:113-6.)

The design and use of allele-specific primers for analyzing polymorphisms are described, for example, in WO 93/22456. Briefly, allele-specific primers are designed to hybridize to a site on target DNA overlapping a polymorphism and to prime DNA amplification according to standard PCR protocols only when the primer exhibits perfect complementarity to the particular allelic form. A single-base mismatch prevents DNA amplification and no detectable PCR product is formed. The method works particularly well when the polymorphic site is at the extreme 3 ‘-end of the primer, because this position is most destabilizing to elongation from the primer.

The primers, once selected, can be used in standard PCR protocols in conjunction with another common primer that hybridizes to the upstream non-coding strand of the RORA gene at a specified location upstream from the polymorphism. The common primers are chosen such that the resulting PCR products can vary from about 100 to about 300 bases in length, or about 150 to about 250 bases in length, although smaller (about 50 to about 100 bases in length) or larger (about 300 to about 500 bases in length) PCR products are possible. The length of the primers can vary from about 10 to 30 bases in length, or about 15 to 25 bases in length.

Primers or probes can be labeled by incorporating a label detectable by spectroscopic, photochemical, biochemical, immunochemical, radiological, radiochemical or chemical means. Useful labels include ³²P, fluorescent dyes, electron-dense reagents, enzymes, biotin, or haptens and proteins for which antisera or monoclonal antibodies are available.

Many of the methods for detecting polymorphisms involve amplifying DNA or RNA from target samples (e.g., amplifying the segments of the RORA gene of an individual using RORA-specific primers) and analyzing the amplified gene segments. This can be accomplished by standard polymerase chain reaction (PCR & RT-PCR) protocols or other methods known in the art, and described in U.S. Pat. Nos. 4,683,195; 4,683,202; and 4,965,188; each incorporated herein by reference. Other suitable amplification methods include the ligase chain reaction (Wu and Wallace (1988) GENOMICS 4:560-569); the strand displacement assay (Walker et al. (1992) PROC. NATL. ACAD. SCI. USA 89:392-396, Walker et al. (1992) NUCL. ACIDS RES. 20:1691-1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al. (1989) PROC. NATL. ACAD. SCI. USA 86:1173-1177); and self-sustained sequence replication (3SR) (Guatelli et al. (1990) PROC. NATL. ACAD. SCI. USA 87:1874-1878 and WO 92/08800); each incorporated herein by reference. Alternatively, methods that amplify the probe to detectable levels can be used, such as QB-replicase amplification (Kramer et al. (1989) NATURE, 339:401-402, and Lomeli et al. (1989) CLIN. CHEM. 35:1826-1831, both of which are incorporated herein by reference). A review of known amplification methods is provided in Abramson et al. (1993) CURRENT OPINION IN BIOTECHNOLOGY 4:41-47, incorporated herein by reference.

Amplification products generated using any of the above methods can be analyzed by the use of denaturing gradient gel electrophoresis. Different alleles can be identified based on sequence-dependent melting properties and electrophoretic migration in solution. See Erlich, ed., PCR Technology, Principles and Applications for DNA Amplification, Chapter 7 (W.H. Freeman and Co, New York, 1992). Upon generation of an amplified product, polymorphisms of interest can be identified by DNA sequencing methods, such as the chain termination method (Sanger et al. (1977) PROC. NATL. ACAD. SCI. 74:5463-5467) or PCR-based sequencing. See Sambrook et al., MOLECULAR CLONING, A LABORATORY MANUAL (2nd Ed., CSHP, New York 1989) and Zyskind et al., RECOMBINANT DNA LABORATORY MANUAL (Acad. Press, 1988).

Other useful analytical techniques that can detect the presence of a polymorphism in the amplified product include single-strand conformation polymorphism (SSCP) analysis, denaturing gradient gel electropohoresis (DGGE) analysis, and/or denaturing high performance liquid chromatography (DHPLC) analysis. In such techniques, different alleles can be identified based on sequence- and structure-dependent electrophoretic migration of single stranded PCR products. Amplified PCR products can be generated according to standard protocols, and heated or otherwise denatured to form single stranded products, which may refold or form secondary structures that are partially dependent on base sequence. An alternative method, referred to herein as a kinetic-PCR method, in which the generation of amplified nucleic acid is detected by monitoring the increase in the total amount of double-stranded DNA in the reaction mixture, is described in Higuchi et al. (1992) BIO/TECHNOLOGY, 10:413-417, incorporated herein by reference.

Polymorphic variant detection can also be accomplished by direct PCR amplification, for example, via Allele-Specific PCR (AS-PCR) which is the selective PCR amplification of one of the alleles to detect a polymorphic variant (e.g., a SNP variant). Selective amplification is usually achieved by designing a primer such that the primer will match/mismatch one of the alleles at the 3′-end of the primer. The amplifying may result in the generation of RORA allele-specific oligonucleotides, which span any of the SNPs, rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570. The RORA-specific primer sequences and RORA allele-specific oligonucleotides may be derived from the coding (exons) or non-coding (promoter, 5′ untranslated, introns or 3′ untranslated) regions of the RORA gene. Polymorphic variant detection also can be accomplished using restriction fragment length polymorphism (RFLP) analysis, where the presence or absence of a particular variant at a polymorphic site creates or eliminates a restriction site for a particular endonuclease, creating a different pattern of fragment lengths, depending upon the variant present, when nucleic acid containing the polymorphic variant is exposed to the endonuclease.

A wide variety of other methods are known in the art for detecting polymorphisms in a biological sample. See, e.g., U.S. Pat. No. 6,632,606; Shi (2002) AM. J. PHARMACOGENOMICS 2:197-205; Kwok et al. (2003) CURR. ISSUES BIOL. 5:43-60). Detection of the single nucleotide polymorphic form (i.e., the presence or absence of the variant at rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570), alone and/or in combination with each other and/or in combination with additional RORA gene polymorphisms, may increase the probability of an accurate diagnosis. In one embodiment, screening involves determining the presence or absence of the variant at rs730754. In another embodiment, screening involves determining the presence or absence of the variant at rs975501. In another embodiment, screening involves determining the presence or absence of the variant at rs12900948. In another embodiment, screening involves determining the presence or absence of the variant at rs782925. In another embodiment, screening involves determining the presence or absence of the variant at rs7177611. In another embodiment, screening involves determining the presence or absence of the variant at rs16943429. In another embodiment, screening involves determining the presence or absence of the variant at rs2414687. In another embodiment, screening involves determining the presence or absence of the variant at rs4335725. In another embodiment, screening involves determining the presence or absence of the variant at rs6494231. In another embodiment, screening involves determining the presence or absence of the variant at rs12916023. In another embodiment, screening involves determining the presence or absence of the variant at rs4583176. In another embodiment, screening involves determining the presence or absence of the variant at rs8034864. In another embodiment, screening involves determining the presence or absence of the variant at rs1403737. In another embodiment, screening involves determining the presence or absence of the variant at rs12591914. In another embodiment, screening involves determining the presence or absence of the variant at rs7495128. In another embodiment, screening involves determining the presence or absence of the variant at rs17237514. In another embodiment, screening involves determining the presence or absence of the variant at rs17270640. In another embodiment, screening involves determining the presence or absence of the variant at rs11071570.

In diagnostic methods, the analysis of rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570 can be combined with each other and/or can be combined with analysis of polymorphisms in other genes associated with AMD, detection of protein markers of AMD (see, e.g., U.S. Patent Application Publication Nos. US2003/0017501 and US2002/0102581 and International Patent Application Publication Nos. WO0184149 and WO0106262), assessment of other risk factors of AMD (such as family history), with ophthalmological examination, and with other assays and procedures.

Screening also can involve detecting a haplotype which includes two or more polymorphic variants. Such polymorphic variants include those described herein and/or additional RORA gene polymorphisms, and/or other genes associated with AMD and/or other risk factors. The polymorphic variants include, but are not limited to, those at rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and rs11071570.

For the two or more polymorphic variants, one determines if the risk variant is present or absent (for risk variant polymorphic variants) and/or if the common allele is present or absent (for protective variants) in order to diagnose a subject for being at increased risk of developing AMD. Conversely, for the two or more polymorphic variants, one can determine if the common allele is present or absent (for risk variants) and/or the protective variant is present or absent (for protective variants) in order to diagnose a subject for being at reduced risk of developing AMD. If the subject has a haplotype comprising the common allele (an adenine) in the forward sequence of rs17237514 and the protective variant (an adenine) in the forward sequence of rs4335725, then the subject has a reduced risk of developing AMD relative to a person without the haplotype. If the subject has a haplotype comprising the common allele (an adenine) in the forward sequence of rs12900948, the common allele (an adenine) in the forward sequence of rs730754, and the common allele (a cytosine) in the forward sequence of rs8034864, then the subject has an increased risk of developing AMD relative to a person without the haplotype.

A polymorphic variant (e.g., a SNP variant) either individually or within a genetic profile for AMD as described herein (e.g., rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and/or rs11071570) may be detected directly or indirectly. Direct detection refers to determining the presence or absence of a specific polymorphic variant identified in the genetic profile using a suitable nucleic acid, such as an oligonucleotide in the form of a probe or primer as described above. Alternatively, direct detection can include querying a pre-produced database comprising all or part of the individual's genome for a specific polymorphic variant in the genetic profile. Other direct methods are described herein and are known to those skilled in the art. Indirect detection refers to determining the presence or absence of a specific polymorphic variant identified in the genetic profile by detecting a surrogate or proxy polymorphic variant that is in linkage disequilibrium with the polymorphic variant in the individual's genetic profile. Detection of a proxy polymorphic variant is indicative of a polymorphic variant of interest and is increasingly informative to the extent that the polymorphic variants are in linkage disequilibrium, e.g., at least 50%, 60%, 70%, 80%, 90%, 95%, 98%, or about 100% LD. Another indirect method involves detecting allelic variants of proteins accessible in a sample from an individual that are consequent of a risk-associated or protection-associated allele in DNA that alters a codon.

It is also understood that a genetic profile as described herein may include one or more nucleotide polymorphism(s) that are in linkage disequilibrium with a polymorphism that is causative of disease. In this case, the polymorphic variant in the genetic profile is a surrogate polymorphic variant for the causative polymorphism.

Genetically linked polymorphic variants, including surrogate or proxy polymorphic variants, can be identified by methods known in the art. Non-random associations between polymorphisms (including single nucleotide polymorphisms, or SNPs) at two or more loci are measured by the degree of linkage disequilibrium (LD). The degree of linkage disequilibrium is influenced by a number of factors including genetic linkage, the rate of recombination, the rate of mutation, random drift, non-random mating and population structure. Moreover, loci that are in LD do not have to be located on the same chromosome, although most typically they occur as clusters of adjacent variations within a restricted segment of DNA. Polymorphisms that are in complete or close LD with a particular disease-associated polymorphic variant are also useful for screening, diagnosis, and the like.

C. Protein-Based or Phenotypic Detection of Polymorphism

Where polymorphisms are associated with a particular phenotype, then individuals that contain the polymorphism can be identified by checking for the associated phenotype. For example, where a polymorphism causes an alteration in the structure, sequence, expression and/or amount of a protein or gene product, and/or size of a protein or gene product, the polymorphism can be detected by protein-based assay methods.

Protein-based assay methods include electrophoresis (including capillary electrophoresis and one- and two-dimensional electrophoresis), chromatographic methods such as high performance liquid chromatography (HPLC), thin layer chromatography (TLC), hyperdiffusion chromatography, and mass spectrometry.

Where the structure and/or sequence of a protein is changed by a polymorphism of interest, one or more antibodies that selectively bind to the altered form of the protein can be used. Such antibodies can be generated and employed in detection assays such as fluid or gel precipitin reactions, immunodiffusion (single or double), immunoelectrophoresis, radioimmnunoassay (RIA), enzyme-linked immunosorbent assays (ELISAs), immunofluorescent assays, Western blotting and others.

### III. Kits

In certain embodiments, one or more oligonucleotides of the invention are provided in a kit or on device (e.g., an array) useful for detecting the presence of a predisposing or a protective polymorphism in a nucleic acid sample of an individual whose risk for AMD is being assessed. A useful kit can contain oligonucleotides specific for particular alleles of interest as well as instructions for their use to determine risk for AMD. In some cases, the oligonucleotides may be in a form suitable for use as a probe, for example, fixed to an appropriate support membrane. In other cases, the oligonucleotides can be intended for use as amplification primers for amplifying regions of the loci encompassing the polymorphic sites, as such primers are useful in the preferred embodiment of the invention. Alternatively, useful kits can contain a set of primers comprising an allele-specific primer for the specific amplification of alleles. As yet another alternative, a useful kit can contain antibodies to a protein that is altered in expression levels, structure and/or sequence when a polymorphism of interest is present within an individual. Other optional components of the kits include additional reagents used in the genotyping methods as described herein. For example, a kit additionally can contain amplification or sequencing primers which can, but need not, be sequence-specific, enzymes, substrate nucleotides, reagents for labeling and/or detecting nucleic acid and/or appropriate buffers for amplification or hybridization reactions.

In one embodiment, a kit or device for diagnosing susceptibility to age-related macular degeneration (AMD) in a subject comprising oligonucleotides that distinguish alleles at at least one polymorphic site in the RORA gene associated with risk of developing AMD. The oligonucleotides may distinguish alleles at at least one polymorphic site selected from the group consisting of rs12900948, rs730754, rs8034864, rs17237514 and rs4335725. In an exemplary embodiment, the oligonucleotides are primers for nucleic acid amplification of a region spanning a RORA gene polymorphic site selected from the group consisting of rs12900948, rs730754, rs8034864, rs17237514 and rs4335725. In another exemplary embodiment, the oligonucleotides are probes for nucleic acid hybridization of a region spanning a RORA gene polymorphic site selected from the group consisting of rs12900948, rs730754, rs8034864, rs17237514 and rs4335725.

In certain embodiments, a kit or device may include oligonucleotides that distinguish alleles at more than one polymorphic site in the RORA gene. For example the kit or device may include oligonucleotides that distinguish alleles, for example, at (a) rs12900948, rs730754, rs8034864; and/or (b) rs17237514 and rs4335725.

In still other embodiment, a kit or device may include oligonucleotides that distinguish alleles at rs1061170 (CFH), rs800292 (CFH), rs10490924 (LOC387715) and rs11200638 (ARMS2/HTRA1), or other alleles associated with AMD.

### IV. Analysis Systems and Reports

In a further aspect, the invention provides a system for analyzing one or more SNPs selected from the group of rs730754, rs975501, rs12900948, rs782925, rs7177611, rs16943429, rs2414687, rs4335725, rs6494231, rs12916023, rs4583176, rs8034864, rs1403737, rs12591914, rs7495128, rs17237514, rs17270640, and/or rs11071570 comprising: reagents to detect (directly or indirectly) in a sample from the patient the presence or absence of one or more of the rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and/or rs11071570 SNPs (including the presence or absence of a specific variant at a particular SNP); hardware to perform detection of the SNPs; and a processor to execute stored instruction sequences (for example, software) that analyze the detected information (e.g., to identify and/or calculate a level of one or more SNPs), to determine if the patient is at risk of developing, or has, AMD, and/or to determine if the patient is responsive to a treatment. The reagents to detect one or more of the rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and/or rs11071570SNPs (including the presence or absence of a specific variant at a particular SNP) may be, for example, any of those described herein, including primers, probes, and other molecules that bind to and/or amplify one or more of the rs12900948, rs4335725, rs730754, rs8034864, rs17237514, rs975501, rs782925, rs7177611, rs16943429, rs2414687, rs6494231, rs12916023, rs4583176, rs1403737, rs12591914, rs7495128, rs17270640, and/or rs11071570SNPs (including a specific variant at a particular SNP) and/or a proxy polymorphic site (including a proxy polymorphic variant). The hardware is preferably a machine or computer to perform the detection step, and the processor may be by, for example, part of a computer or machine specifically configured to perform the analysis described herein.

Suitable software and processors are well known in the art and are commercially available. The program may be embodied in software and stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

After detecting (including detecting the presence or absence of) one or more of the rs730754, rs975501, rs12900948, rs782925, rs7177611, rs16943429, rs2414687, rs4335725, rs6494231, rs12916023, rs4583176, rs8034864, rs1403737, rs12591914, rs7495128, rs17237514, rs17270640, and/or rs11071570 SNPs (including the presence or absence of a specific variant at a particular SNP), and producing the assay results, findings, diagnoses, predictions and/or treatment, they are typically recorded and/or communicated to, for example, medical professionals and/or patients. In certain embodiments, the assay results, findings, diagnoses, predictions and/or treatment recommendations are communicated to the patient, directly, or to the patient's treating physician, after the assay and analysis is completed. The assay results, findings, diagnoses, predictions and/or treatment recommendations may be communicated to medical professionals and/or patients by any means of communication, such as a written report (e.g., on paper), an auditory report, or an electronic record.

Communication may be facilitated by use electronic forms of communication and/or by use of a computer, such as in case of email or telephone communications. In certain embodiments, the communication containing assay results, findings, diagnoses, predictions and/or treatment recommendations may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing/prognosing of diseases, and communicating of assay results, findings, diagnoses, predictions and/or treatment recommendations, may be carried out in diverse (e.g., foreign) jurisdictions. For example, in some embodiments the assays are performed, or the assay results analyzed, in a country or jurisdiction which differs from the country or jurisdiction to which the assay results, findings, diagnoses, predictions and/or treatment recommendations are communicated.

To facilitate diagnosis, the presence, absence, and/or level of one or more of the rs730754, rs975501, rs12900948, rs782925, rs7177611, rs16943429, rs2414687, rs4335725, rs6494231, rs12916023, rs4583176, rs8034864, rs1403737, rs12591914, rs7495128, rs17237514, rs17270640, and/or rs11071570 SNPs (including the presence, absence, and/or level of a specific variant at a particular SNP) and/or of a proxy polymorphic site (including the presence, absence, and/or level of a proxy polymorphic variant) can be displayed on a display device or contained electronically or in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

The methods of this invention, when practiced for commercial diagnostic purposes, generally produce a report or summary of the presence or absence of one or more of the SNPs described herein (including the presence or absence of a specific variant at a particular SNP) and/or a proxy polymorphic site (including the presence or absence of a proxy polymorphic variant). The methods of this invention also can produce a report comprising one or more predictions and/or diagnoses concerning a patient, for example whether the patient is at risk of developing, or has, neovascular AMD.

The methods and reports of this invention can further include storing the report in a database. Alternatively, the method can further create a record in a database for the subject and populate the record with data. Reports can include a paper report, an auditory report, or an electronic record. It is contemplated that the report is provided to a physician and/or the patient. The receiving of the report can further include establishing a network connection to a server computer that includes the data and report and requesting the data and report from the server computer. The methods provided by the present invention may also be automated in whole or in part.

In another aspect, the invention provides an article of manufacture having a computer-readable medium with computer-readable instructions embodied thereon for performing the methods and implementing the systems described herein. In particular, the stored instruction sequences of the present invention may be embedded on a computer-readable medium, such as, but not limited to, a floppy disk, a hard disk, an optical disk, a magnetic tape, a PROM, an EPROM, CD-ROM, or DVD-ROM or downloaded from a server. The stored instruction sequences may be embedded on the computer-readable medium in any number of computer-readable instructions, or languages such as, for example, FORTRAN, PASCAL, C, C++, Java, C#, Tc1, BASIC and assembly language. Further, the computer-readable instructions may, for example, be written in a script, macro, or functionally embedded in commercially available software (such as, e.g., EXCEL or VISUAL BASIC).

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

In light of the foregoing description, the specific non-limiting examples presented below are for illustrative purposes and not intended to limit the scope of the invention in any way.

### EXAMPLES

### Example 1

### Variants in the RORA Gene Alter the Risk of Neovascular AMD

This Example describes the elucidation of alleles either conferring protection to, or increasing the risk of, the development of AMD. It also identifies the biological relevance of polymorphic variants in the RORA gene, particularly in intron 1 of the RORA gene.

Previous studies of linkage, genome-wide association studies (GWAS) and gene expression, as described in International Patent Application PCT/US2009/040220, led to the discovery of the candidate gene retinoic acid receptor-related orphan receptor α (RORA). RORA may be an indirect regulator of angiogenesis, as well as function in oxidative stress, immunity/inflammation and lipid transport, processes that have been implicated in the pathophysiology of neovascular AMD. (Anderson et al. (2002) AM. J. OPHTHALMOL. 134:411-431; Besnard et al. (2002) FEBS LETT. 511(1-3):36-48; Besnard et al. (2001) CIRC. RES. 89(12):1209-1215; Boukhtouche et al. (2004) ARTERIOSCLER. THROMB. VASC. BIOL. 24(4):637643; Boukhtouche et al. (2006) J. NEUROCHEM. 96(6):1778-1789; Conley et al. (2005) HUM. MOL. GENET. 14(14):1991-2002; Hageman et al. (2001) PROG. RETIN. EYE RES. 20(6):705-732; Johnson (2005) NUTR. REV. 63:9-15; Klein et al. (2003) ARCH OPHTHALMOL. 121:1151-1155; Lau et al. (2008) J. BIOL. CHEM. 283(26):18411; Zhu et al. (2006) ONCOGENE 25(20):2901.) Recently, Fujieda and colleagues showed that RORA regulates cone genes (Crx, Arr3), during mouse retinal development (Fujieda et al. (2009) J. NEUROCHEM. 108(1):91).

The protocol described herein was reviewed and approved by the Institutional Review Boards at Massachusetts Eye and Ear Infirmary, Boston, Mass. and conforms to the tenets of the Declaration of Helsinki. Eligible patients were enrolled in this study after they gave informed consent, either in person, over the phone, or through the mail, before completing a standardized questionnaire and donating 10-50 ml of venous blood.

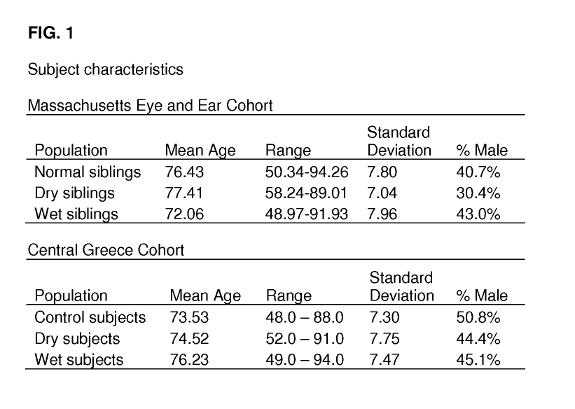

Sibling pairs, mainly of European ancestry, were recruited for this study. The population characteristics of the sibling pairs are summarized in FIG. 1 (DeAngelis, et al. (2008) OPHTHALMOL, 115, 1209-1215). In brief, all index patients were aged 50 years or older, except where one individual was 49 years of age, and had the neovascular form of AMD in at least one eye, defined by subretinal hemorrhage, fibrosis, or fluorescein angiographic presence of neovascularization documented at the time of, or prior to, enrollment in the study. Patients whose only exudative finding was a retinal pigment epithelium detachment were excluded because this finding may not represent definite neovascular AMD. Patients with signs of pathologic myopia, presumed ocular histoplasmosis syndrome, angioid streaks, choroidal rupture, any hereditary retinal diseases other than AMD, and previous laser treatment due to retinal conditions other than AMD were also excluded.

A. Family Patient Populations

Of the sibling pairs analyzed herein, 150 were extremely phenotypically discordant. Extremely discordant sibling pairs include one unaffected sibling who had normal maculae at an age older than that at which the index patient (the affected sibling) was first diagnosed with neovascular AMD. Normal maculae (defined as the zone centered at the foveola and extending two disk diameters, or 3000 μm, in radius) fulfilled the following criteria: 0-5 small drusen (all less than 63 μm in diameter), no retinal pigment epithelium (RPE) abnormalities, no geographic atrophy, and no neovascularization [as defined previously; AMD “category 1 or less” on the Age-Related Eye Disease Study (AREDS) scale (AREDS Research Group. (2000) AREDS Report No. 3, pp. 2224-2232)]. Disease status of every participant was confirmed by at least two of the investigators by evaluation of fundus photographs or fluorescein angiograms except when one of the investigators directly examined an unaffected sibling during a home visit (n=4 cases). Smoking data, as measured in pack years, was available for every participant.

An additional 46 discordant sibling pairs were analyzed where each pair was comprised of one sibling (the index sibling) with neovascular AMD and the other sibling (the control sibling) with mild or very early AMD [AREDS category 2 (AREDS Research Group. (2000) AREDS Report No. 3, pp. 2224-2232)] at 65 years of age or older in most cases. Siblings were categorized as early AMD only if they met the following criteria for the definition of AREDS category 2: small (<63 μm) drusen with total area greater than or equal to a 125 μm diameter circle, or at least one intermediate drusen (≧63 and <125 μm) or presence of pigment. These criteria are based on published epidemiologic studies that indicate that elderly individuals with such maculae rarely go onto develop neovascular AMD during a 10 year follow-up (Klein, et al. (2002) AM. J. EPIDEMIOL., 156, 589-598).

A total of 196 sibling pairs were initially recruited, 150 of which were extremely discordant sibling pairs (i.e., one sibling with neovascular AMD and one normal sibling—AREDS category 1 or less) and the remaining 46 were discordant, meaning one sibling had neovascular AMD and the other sibling had early dry, AMD (AREDS category 2) (FIG. 1). The mean±SD age of the affected siblings (neovascular AMD) was 72.1±8.0 years (age range, 49.0-92.0 years), the age of the mildly affected siblings (dry AMD) was 77.4±7.0 years (age range, 58.2-89.1 years), and the age of the unaffected siblings was 76.4±7.8 years (age range, 50.3-94.3 years). Forty-one percent of the unaffected siblings, 30% of the dry AMD siblings and 43% of the matching affected siblings were male. All participants were white and mostly of European descent.

B. Unrelated Case and Control Population

The findings observed in the family patient population described above were further investigated in an unrelated population. A replication of the family patient population study was performed on an unrelated case-control cohort from Central Greece that included patients without AMD, with early and intermediate dry AMD [AREDS category 2 (as described above) and AREDS category 3 (n=84); intermediate drusen comprising total area ≧360 μm diameter circle in the presence of soft drusen or ≧656 μm diameter circle in absence of soft drusen, or at least one large drusen (≧125 μm), or non-Central geographic atrophy (AREDS Research Group. (2000) AREDS Report No. 3, pp. 2224-2232)] and with neovascular AMD (cases, n=139) (FIG. 1). These patients were recruited from the medical retina outpatient clinic at the University Hospital of Larissa, Greece. The diagnosis of macular degeneration was confirmed by optical coherence tomography and fluorescein angiography. Color fundus photographs and indocyanine green angiography were performed in some cases.

The cohort from Central Greece consisted of a total of 344 patients: 121 normal controls, 84 early dry patients (AREDS categories 2 and 3), and 139 neovascular patients (FIG. 1). The mean±SD age of the affected subjects (neovascular AMD) was 76.2±7.4 years (age range, 49.0-94.0 years), the age of the mildly affected subjects (dry AMD) was 74.5±7.8 years (age range, 52.0-91.0 years), and the age of the unaffected subjects was 73.5±7.3 years (age range, 48.0-88.0 years). Fifty-one percent of the unaffected subjects, 44% of the AREDS category 2 and AREDS category 3 AMD subjects and 45% of the neovascular subjects were male. All participants were white and from Central Greece.

C. Genotyping Analysis

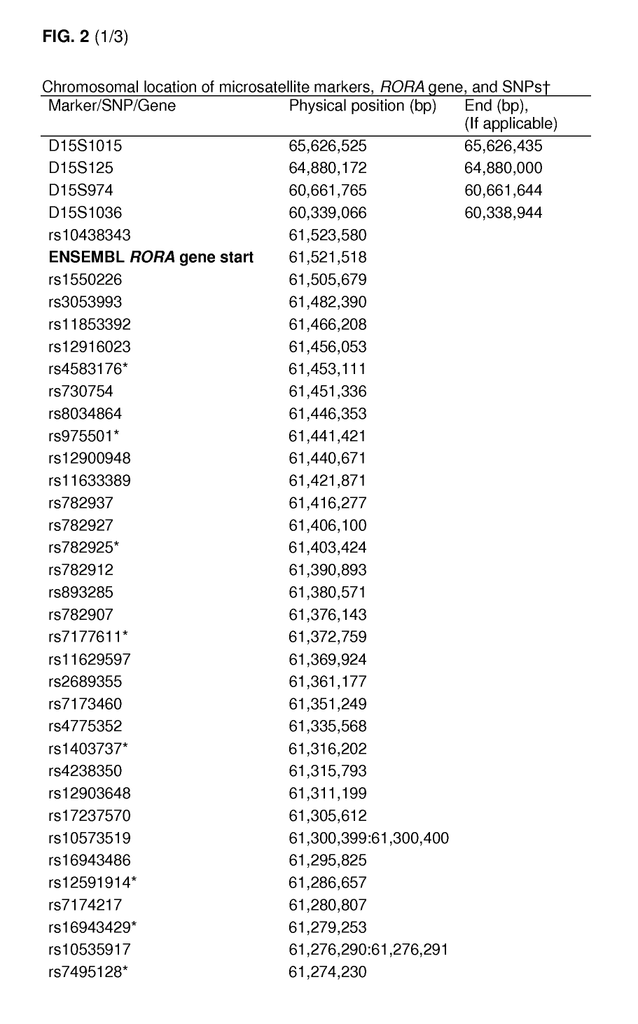

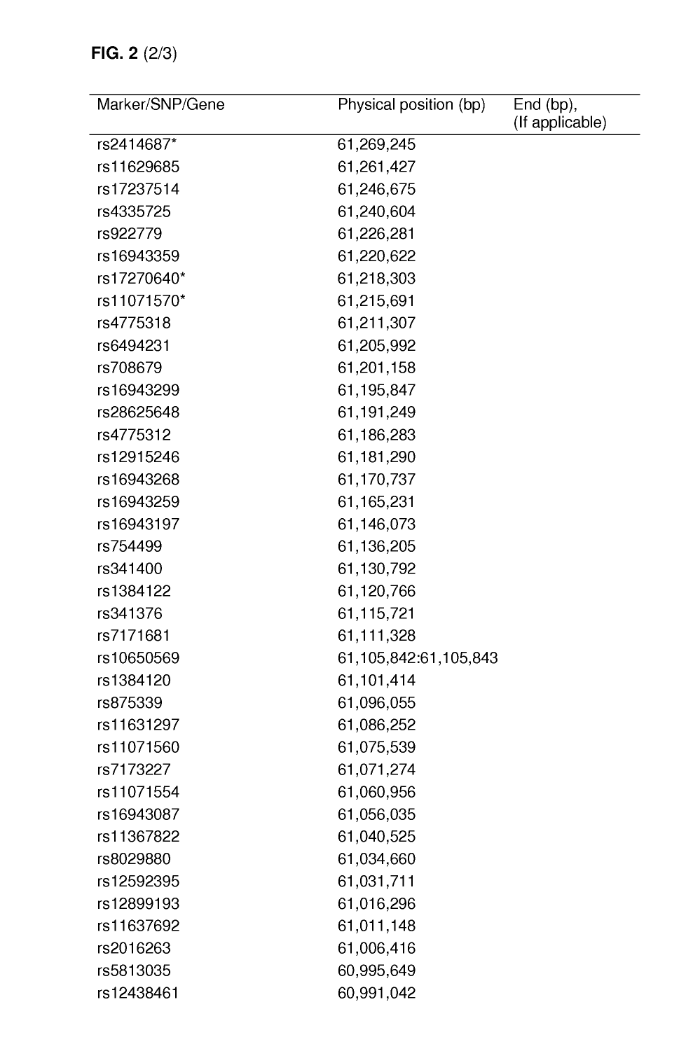

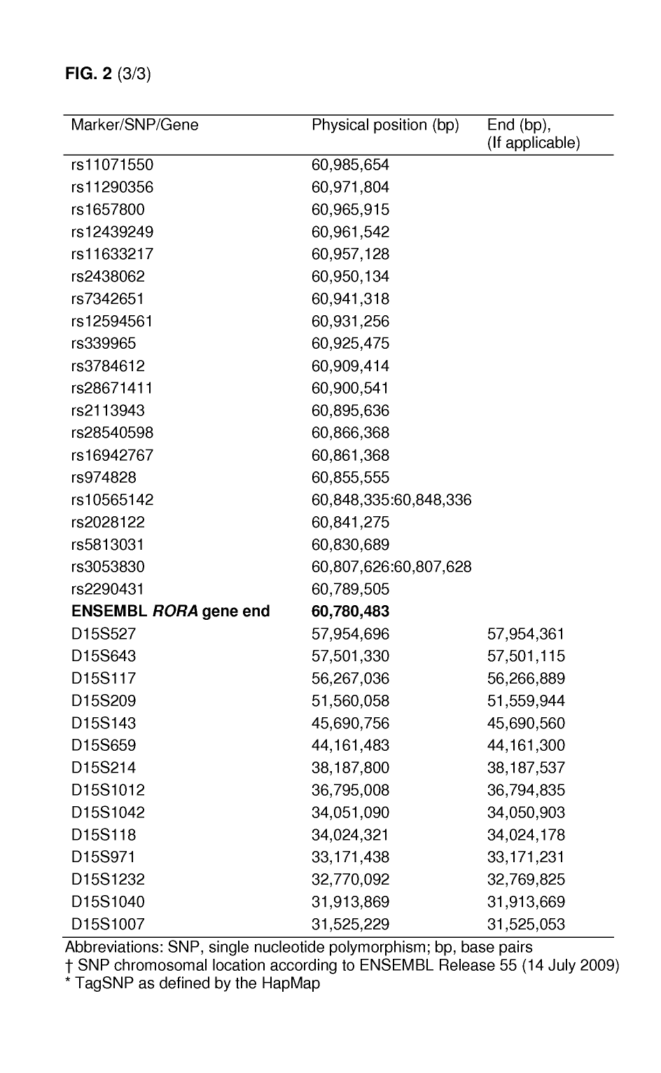

As described above, RORA is located on chromosome 15q and spans approximately 730 kilobases. The SNP location in terms of the largest of four transcripts, which encodes 12 exons as shown in ENSEMBL (RORA-001: ENST00000335670) (available at www.ensembl.org/index.html), were analyzed. Single nucleotide polymorphisms (SNPs) were chosen for analysis at approximately every 5000 base pairs (when variation information was available) in an effort to represent the entire variation within the gene (FIG. 2). Based on the location of SNPs and haplotypes found to be significantly associated with neovascular AMD, 11 TagSNPs were chosen for genotyping using the HapMap (available at www.hapmap.org) and meeting the following criteria: (1) a minor allele frequency greater than 10% and (2) an r² value that was at least 0.8 (FIG. 2).

Multiplex PCR assays were designed using Sequenom SpectroDESIGNER software (version 3.0.0.3) (Sequenom, San Diego, Calif.) by inputting sequence containing the SNP site and 100 base pairs of flanking sequence on either side of the SNP. Briefly, 10 ng genomic DNA was amplified in a 5 μl reaction containing 1× HotStar Taq PCR buffer (Qiagen, Valencia, Calif.), 1.625 mM MgCl2, 500 μm each dNTP, 100 nM each PCR primer, 0.5 U HotStar Taq (Qiagen). The reaction was incubated at 94° C. for 15 mM followed by 45 cycles of 94° C. for 20 s, 56° C. for 30 s, 72° C. for 1 mM, followed by 3 mM at 72° C. Excess dNTPs were removed from the reaction by incubation with 0.3 U shrimp alkaline phosphatase (USB Corporation) at 37° C. for 40 mM followed by a 5 mM incubation at 85° C. to deactivate the enzyme. Single primer extension over the SNP was carried out in a final concentration of between 0.625 μm and 1.5 μm for each extension primer (depending on the mass of the probe), iPLEX termination mix (Sequenom), and 1.35 U iPLEX enzyme (Sequenom), and cycled using a two-step 200 short cycles program; 94° C. for 30 s followed by 40 cycles of 94° C. for 5 s, 5 cycles of 52° C. for 5 s, and 80° C. for 5 s, then 72° C. for 3 mM The reaction was then desalted by addition of 6 mg cation exchange resin followed by mixing and centrifugation to settle the contents of the tube. The extension product was then spotted onto a 384 well SpectroCHIP (Sequenom) before being flown in the MALDI-TOF mass spectrometer. Data was collected, real time, using SpectroTYPER Analyzer 3.3.0.15, SpectraAQUIRE 3.3.1.1 and SpectroCALLER 3.3.0.14 (Sequenom). To ensure data quality, genotypes for each subject were also checked manually. For some replicate samples, direct sequencing was performed.

For these reactions, oligonucleotide primers were selected using the Primer3 program (available at www.primer3.sourceforge.net) to encompass the SNP and flanking intronic sequences. All PCR assays were performed using genomic DNA fragments from 20 ng of leukocyte DNA in a solution of 10× PCR buffer containing 25 mM of MgCl2, 0.2 mM each of dATP, dTTP, dGTP, and dCTP, and 0.5 U of Taq DNA polymerase (USB Corporation). Five molar betaine was added to the reaction mix for rs2414687 (Sigma-Aldrich, St. Louis, Mo.). The temperatures used during the polymerase chain reaction were as follows: 95° C. for 5 min followed by 35 cycles of 58° C. for 30 s, 72° C. for 30 s and 95° C. for 30 s, with a final annealing at 58° C. for 1.5 mM and extension of 72° C. for 5 mM For sequencing reactions, PCR products were digested according to manufacturer's protocol with ExoSAP-IT (USB Corporation) then were subjected to a cycle sequencing reaction using the Big Dye Terminator v 3.1 Cycle Sequencing kit (Applied Biosystems, Foster City, Calif.) according to manufacturer's protocol. Products were purified with Performa DTR Ultra 96-well plates (Edge Biosystems, Gaithersburg, Md.) in order to remove excess dye terminators. Samples were sequenced on an ABI Prism 3100 DNA sequencer (Applied Biosystems). Electropherograms generated from the ABI Prism 3100 were analyzed using the Lasergene DNA and protein analysis software (DNASTAR, Inc., Madison, Wis.). Electropherograms were read by two independent evaluators without knowledge of the subject's disease status. All patients were sequenced in the forward direction (5′-3′), unless variants or polymorphisms were identified, in which case confirmation was obtained in some cases by sequencing in the reverse direction.

Testing of association between SNPs and AMD in the 196 sibling pairs was done using the family-based association test (FBAT) (www.biosun1.harvard.edu/˜fbat/fbat.htm). SNPs were tested for association using the minor allele, as defined by the allele occurring less frequently in the unaffected siblings. Alleles were tested under three genetic models: additive, dominant, and recessive. SNPs were only included for analysis in FBAT if the minor allele frequency (MAF) in the affected and unaffected siblings combined was greater than or equal to 5% and the number of informative families was at least four. Linkage disequilibrium (LD) (both r² and D′) between each of the SNPs was determined using Haploview (www.broad.mit.edu/mpg/haploview).

Haplotype blocks were constructed in Haploview (Gabriel et al. (2002) SCIENCE 296(5576):2225) and individual haplotypes were inferred and tested for association using the family based association test (FBAT). FBAT was used to determine the best fit for each genotypic model tested (Additive, Dominant or Recessive). In order to correct for multiple testing in FBAT, the permutation test was used to examine each of the resulting haplotypes. Genotype and allele frequencies for all SNPs identified as significant, either individually or as part of a haplotype, were calculated in affected and separately in unaffected individuals. Deviation from Hardy-Weinberg Equilibrium was tested on each SNP using the chi square test.

Risk factors were controlled for using conditional logistic regression performed using SAS (SAS, version 9.1; SAS Institute Inc., Cary, N.C.). Included in this analysis were those SNPs identified as significantly associated with neovascular AMD and known risk factors. Known risk factors for the extremely discordant sibling pair cohort included smoking history and the genetic variants CFH rs1061170 (Y402H) and ARMS2/HTRA1 rs10490924/rs11200638 (DeAngelis, et al. (2008) OPHTHALMOL, 115, 1209-1215; Zhang, et al. (2008) BMC MED. GENET., 9, 51). Conditional logistic regression was also used to test gene—gene interaction between RORA SNPs identified as significantly associated with AMD and the CFH and the ARMS2/HTRA1 loci individually. Similarly, conditional logistic regression was used to test gene—environment interaction between RORA and smoking.

For replication in the unrelated case-control cohort from Central Greece, single SNP analysis and haplotype analysis was performed using unconditional logistic regression (UCLR) in SAS (SAS, version 9.1; SAS Institute Inc., Cary, N.C.) under the same three genetic models: additive, dominant, and recessive. Linkage disequilibrium was also tested using Haploview.

D. Analysis of RORA SNPS and Haplotypes in the Extremely Discordant Sibling Pair Cohort

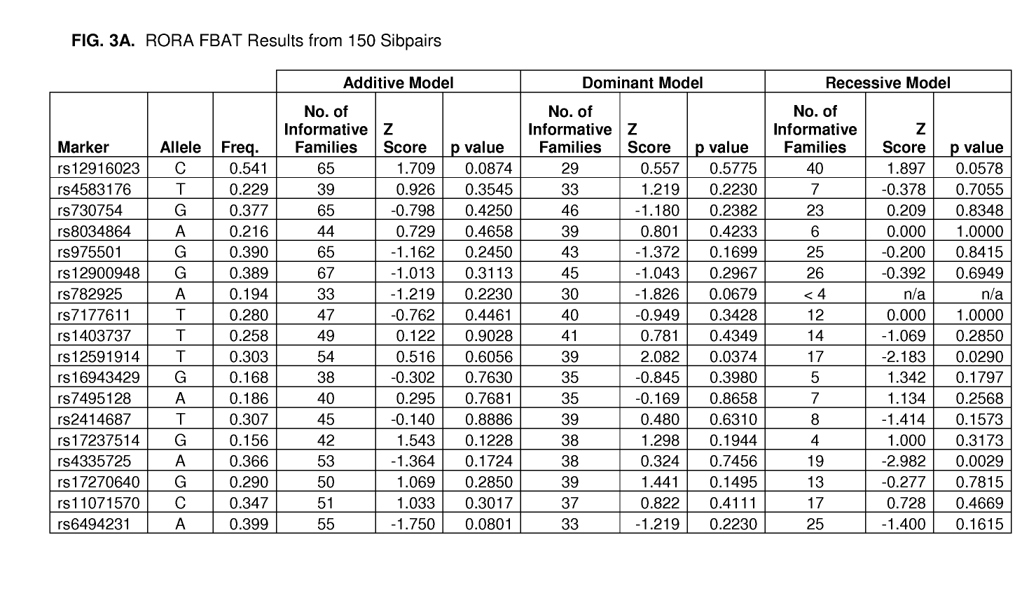

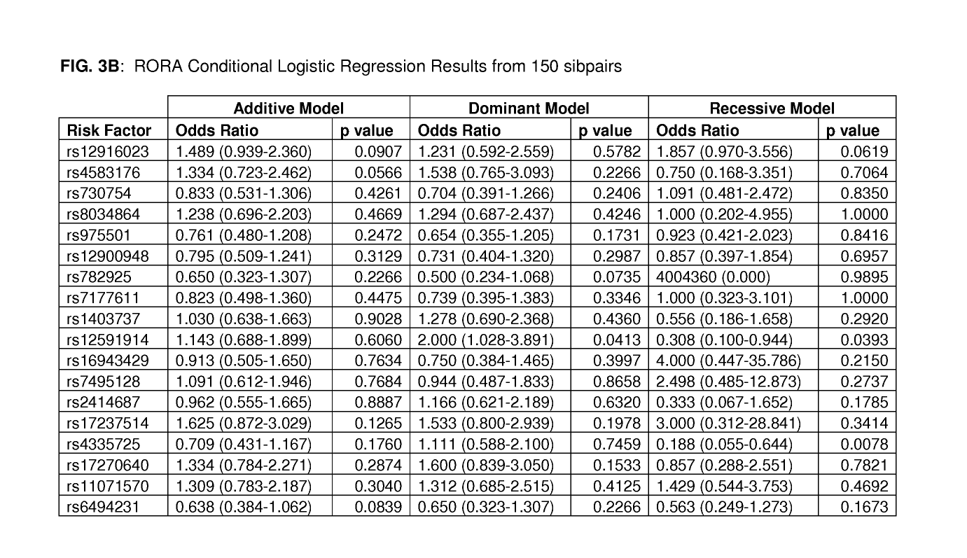

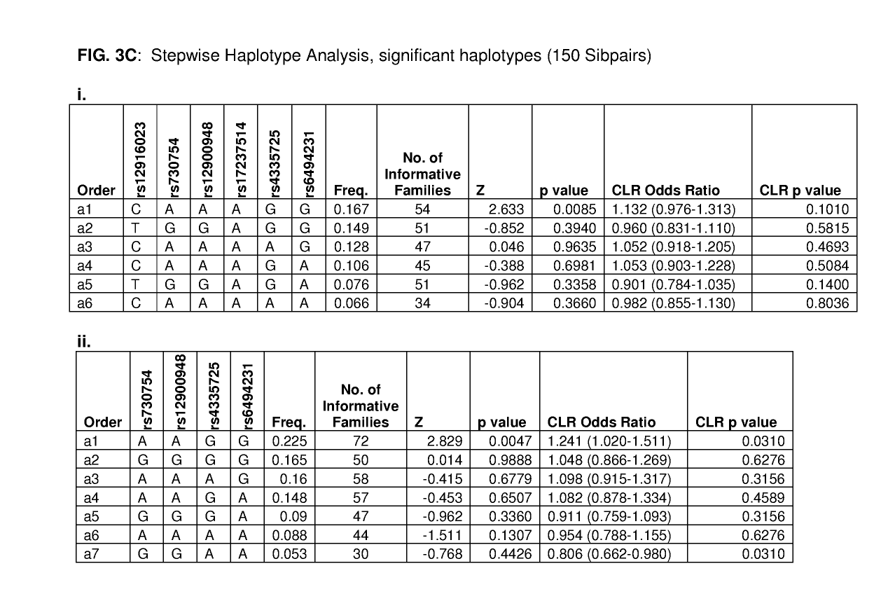

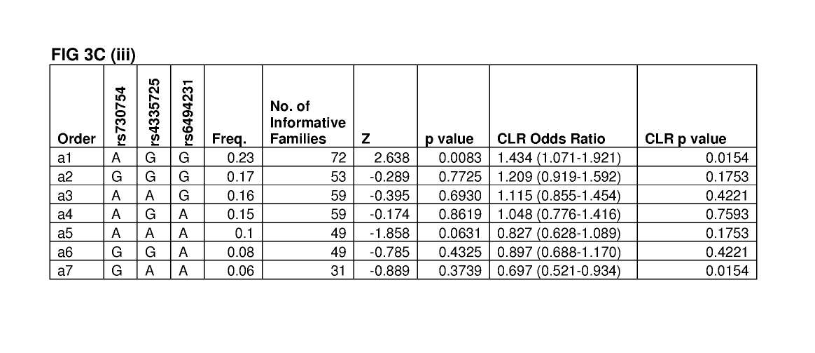

In total, 92 SNPs in the RORA gene were identified as demonstrating variation in the extremely discordant sibling pair cohort (also referred to herein as the discovery cohort) (n=150 pairs; 300 subjects where one sibling had neovascular AMD and one paired sibling did not have AMD) (for SNPs and location refer to FIG. 2). No significant deviations from Hardy-Weinberg equilibrium for any of the variants studied were observed in either affected or unaffected siblings, suggesting unlikely contamination of our dataset. Single SNP analysis using FBAT in the discovery cohort identified rs12591914 and rs4335725 variants located within intron 1 of the RORA-001 transcript (ENST00000335670), as modestly (p=0.029) and significantly (p=0.0029) associated under a recessive model respectively (FIG. 3A). The “allele” column in FIG. 3A corresponds to the minor allele, which is either the risk or protective variant for each of the polymorphic sites, as described herein. Single SNP analysis was conducted using conditional logistic regression (CLR) (FIG. 3B) The RORA SNP rs4335725 remained significant (p≦0.007) after controlling for the following factors: CFH rs1061170 (Y402H), ARMS2/HTRA1 rs10490924/rs11200638, and smoking history in a multiple logistic regression model; however, the rs12591914 SNP did not remain significant.

As shown in FIG. 3C, haplotype analysis on the resulting data was done in a stepwise manner using a combination of FBAT and conditional logistic regression. For haplotype association analysis, all SNPs were entered into the model first and then taken out one by one in order to determine the most significantly parsimonious haplotype. SNP elimination was done using the frequency and preliminary p value given by FBAT. Once the most significant haplotype was identified, it was tested in conditional logistic regression (SAS, 9.0) to determine the corresponding odds ratio and confidence interval. Haplotypes were tested for their association with neovascular AMD as a continuous variable, using the sum of the number of specified alleles in each genotype at each SNP.

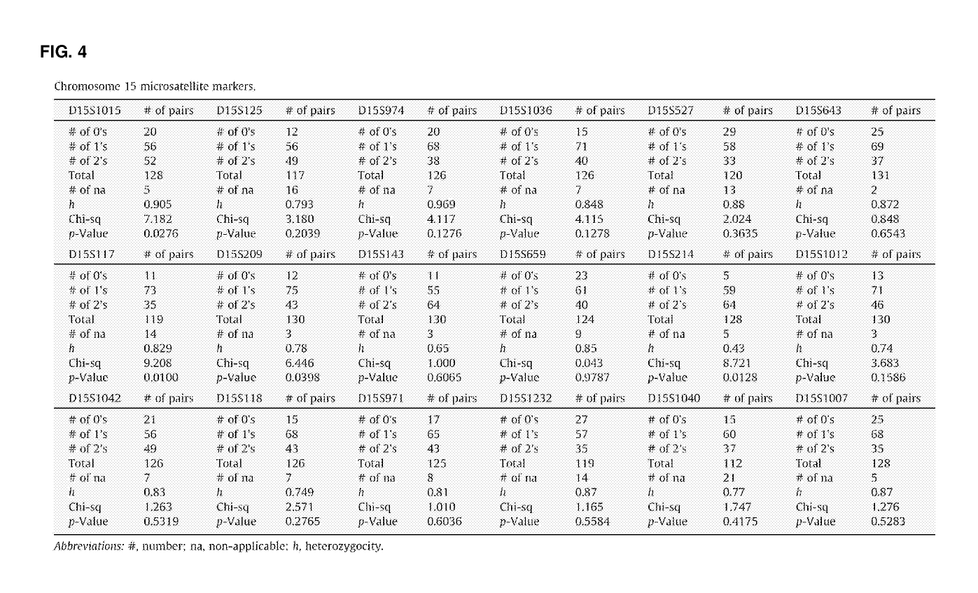

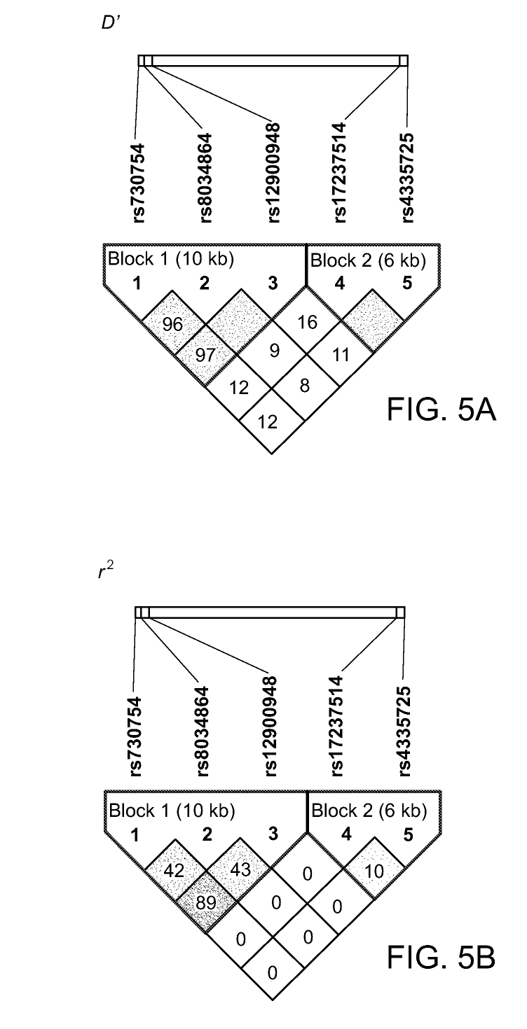

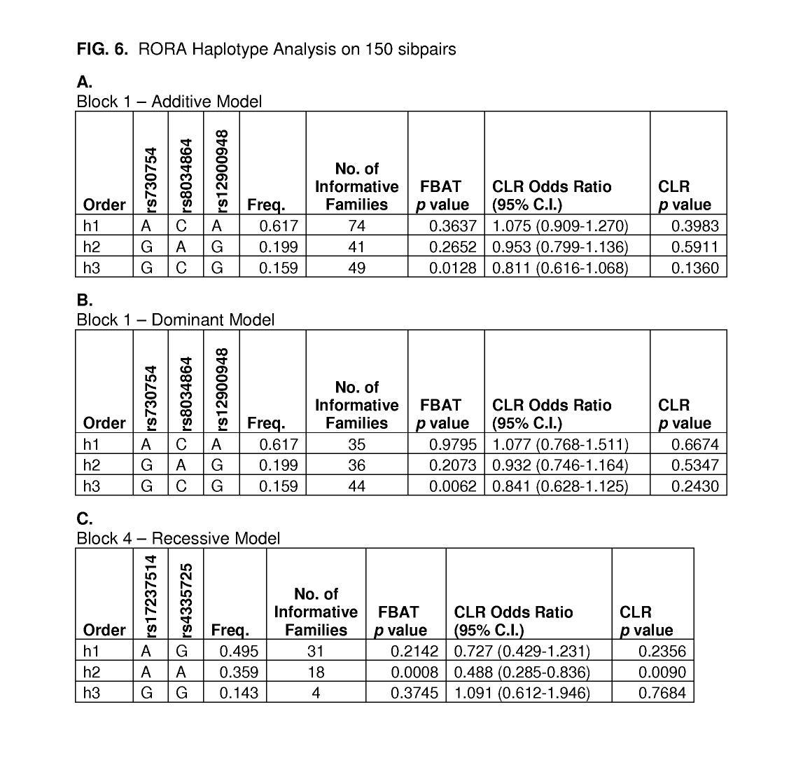

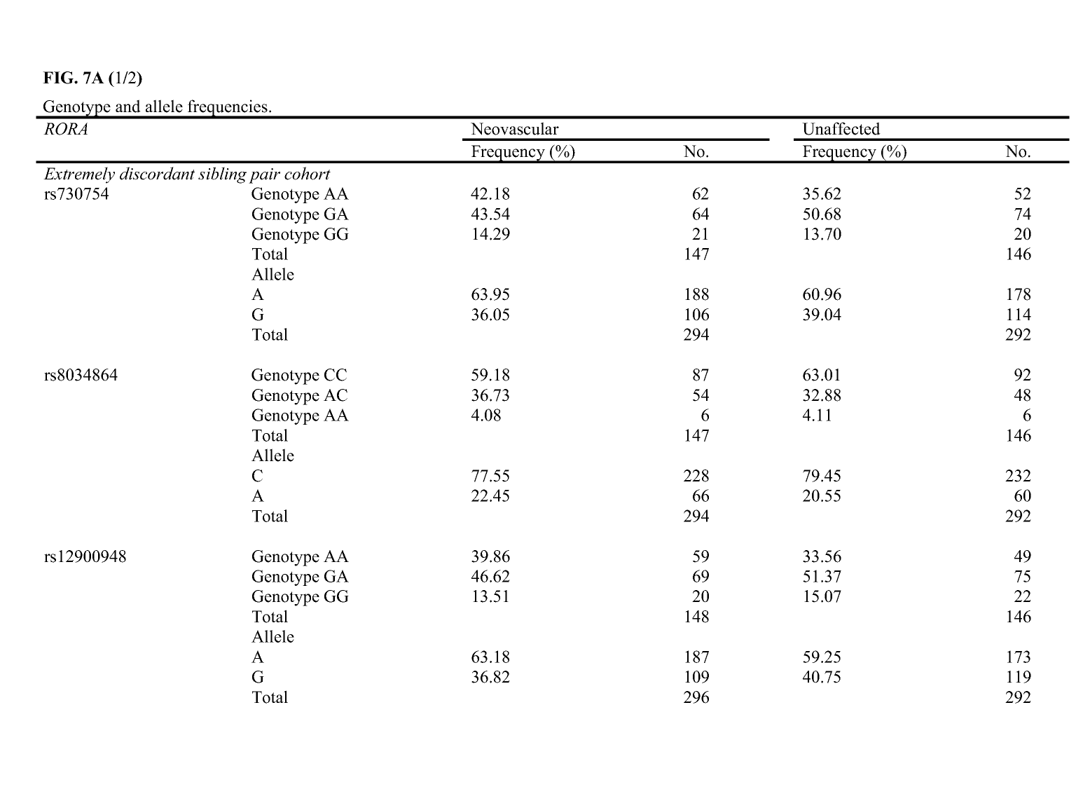

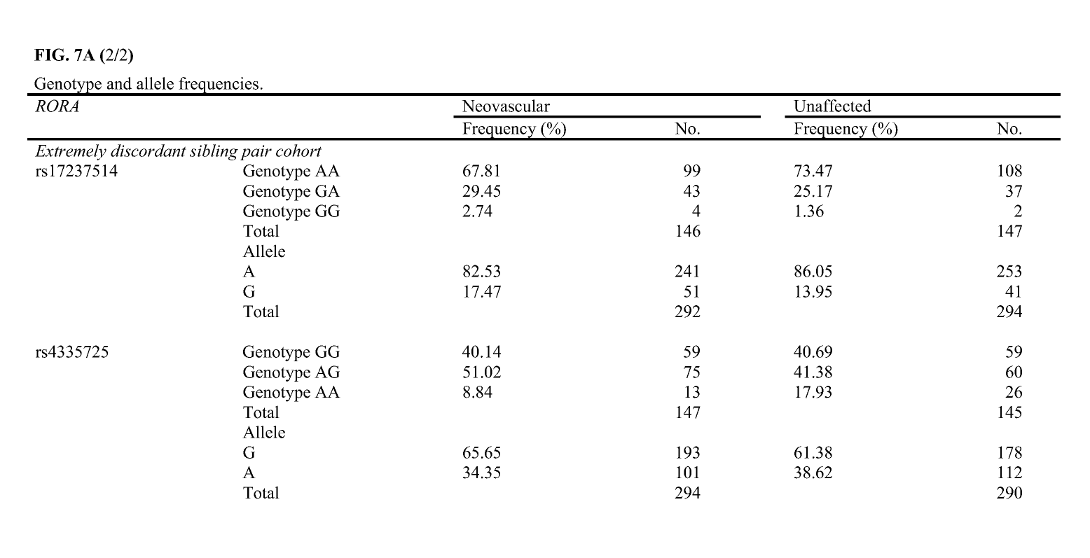

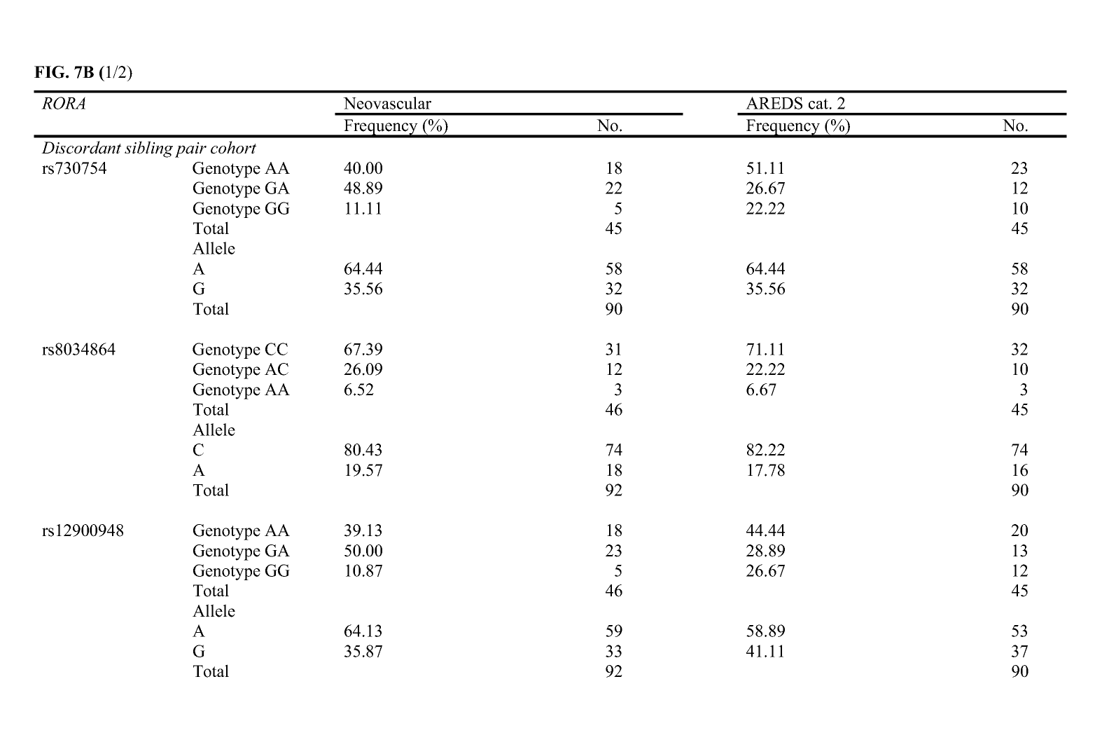

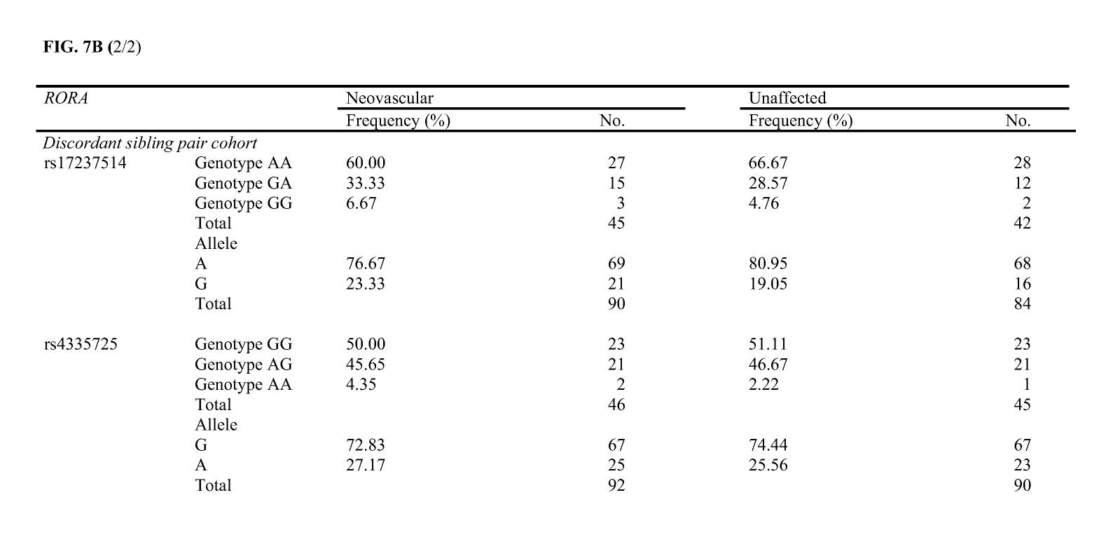

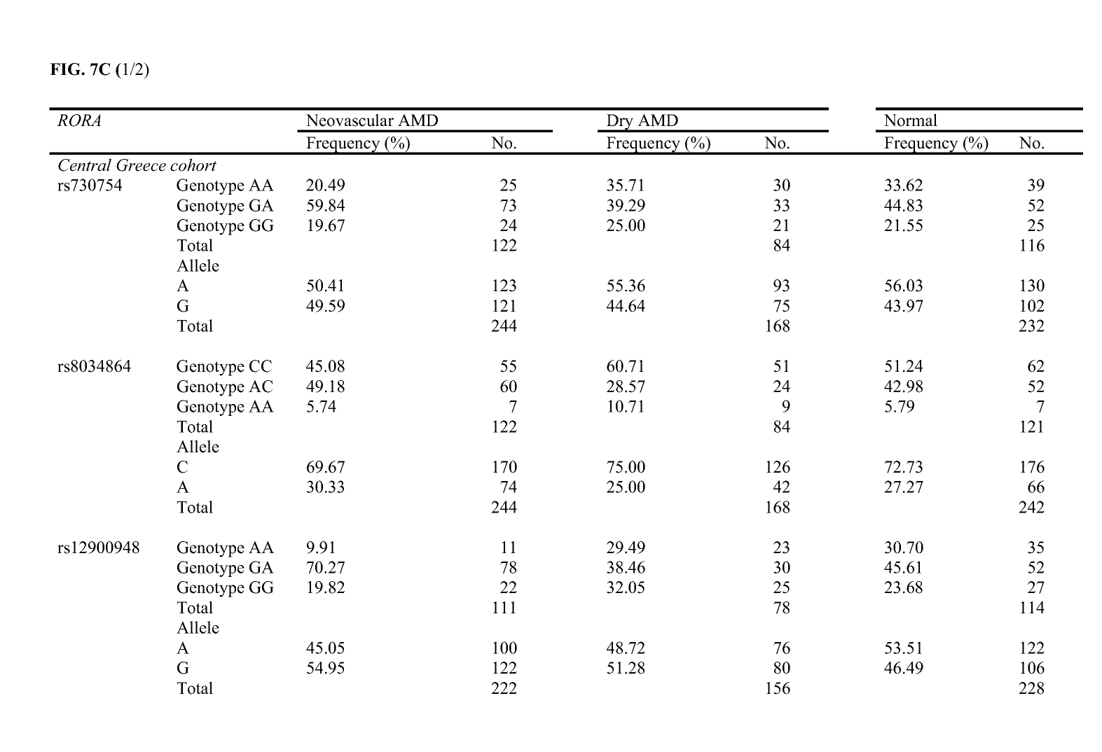

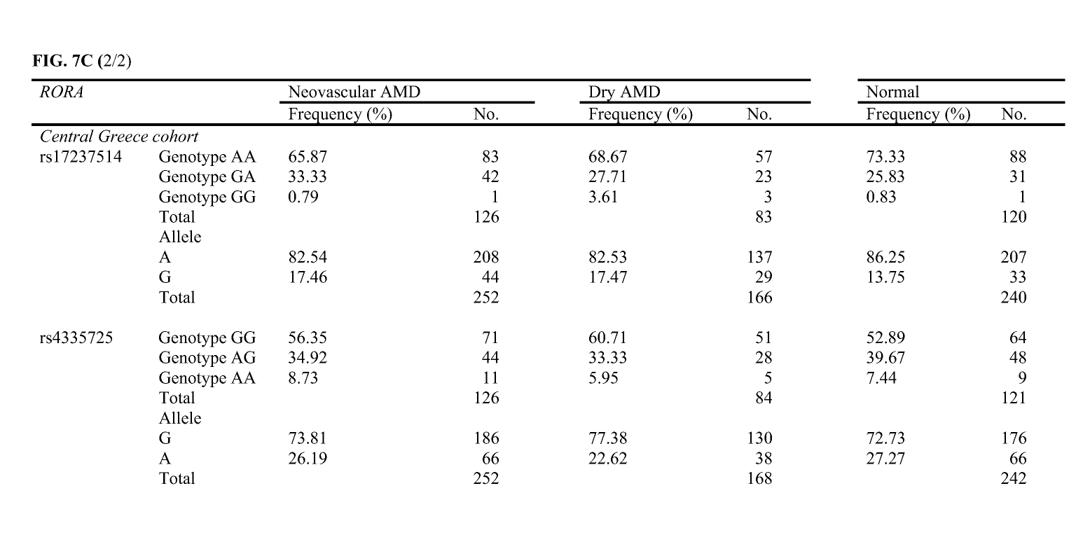

Haploview, using haplotype blocks defined by the Gabriel rule (Gabriel, et al. supra), demonstrated that there were 13 haplotype blocks spanning the more than 700,000 base pairs covered by genotyping (FIG. 4). Of the 13 haplotype blocks, two blocks, both located in intron 1 and comprising three haplotypes, were shown to be significantly associated with neovascular AMD risk in FBAT [h3 in block 1 (GCG) under both additive (p=0.0128) and dominant (p=0.0062) genetic models, as well as h2 in block 4 (AA) under a recessive (p=0.0008) genetic model (FIG. 5-6)]. The overall permutations for the first haplotype in block 1 under both an additive and a dominant model was p=0.03 and p=0.02, respectively. The overall permutation for the second haplotype block was p=0.0018 under a recessive model. Conservation analysis showed that the SNPs comprising these two haplotypes blocks (rs730754, rs8034864, rs12900948, rs17237514 and rs4335725) (FIG. 5) are all well conserved in both rat and mouse. Genotype and allele frequencies for these five SNPs are given in FIG. 7.

Based on these findings, eleven tagging SNPs within intron 1 were chosen to further capture variation and refine this region, which is approximately 550 kilobases. Statistical analysis using FBAT showed that none of the RORA intron 1 tagging SNPs were informative individually or as part of a haplotype as they did not remain significantly associated with neovascular AMD after correction for multiple testing.

As discussed above, two individual SNPs (rs12591914 and rs4335725) showed significant association with AMD risk individually. Five SNPs (rs730754, rs8034864, rs12900948, rs17237514, and rs4335725), comprising 2 blocks (FIG. 6A-C), showed significant association with AMD risk as part of a haplotype. Estimated haplotypes with allele frequency greater than 0.05 were tested for association. When considering all possible haplotypes together, the resulting p value from 100,000 permutations was 0.0018. One significantly associated haplotype, under a recessive genetic model (as shown in FIG. 6C, haplotype h2, AA), contained the significantly associated SNP (rs4335725, p=10 ⁻³). Additionally, the SNPs of haplotype h2 are in linkage disequilibrium and define a region of interest relating both to causative polymorphic sites (as well as causative polymorphic variants) as well as to proxy polymorphic sites (as well as proxy polymorphic variants) for the two polymorphic sites of haplotype h2 (and vice versa). The other haplotypes described herein are similarly definitive of such a region. Twelve other SNPs (rs975501, rs782925, rs7177611, rs16943429, rs2414687, rs6494231, rs12916023, rs4583176, rs1403737, rs7495128, rs17270640, and rs11071570) are associated with AMD risk by virtue of the fact that they cluster with the above SNPs that are individually significantly associated with AMD or significantly associated with AMD as part of a haplotype. All eighteen SNPs are located in intron 1.

Multiple conditional logistic regression (MCLR) analyses were conducted to determine how this haplotype (h2, AA, in FIG. 6C) contributed to the risk of neovascular AMD while adjusting for CFH Y402H, LOC387715 rs10490924 and HTRA1 rs11200638 genotypes as well as smoking. This haplotype, h2, AA, remained significant even after controlling for these known risk factors (P=0.01). The second haplotype block, which contained 3 SNPs and spanned approximately 10,000 by of the RORA promoter, was found significant under an additive model in FBAT (p=0.01) but not in CLR (p=0.1) after correction for multiple testing (FIG. 6A).

E. Analysis of RORA SNPs and Haplotypes in the AREDS Category 2 Discordant Sibling Pair Cohort

The five SNPs that comprised the two haplotype blocks containing significant haplotypes in the discovery cohort (rs730754, rs8034864, rs12900948, for block 1 and rs17237514 and rs4335725 for block 4) were tested for association in the AREDS category 2 discordant sibling pair cohort where the index patient had the neovascular form of AMD and the matched sibling had the early/dry form of AMD (AREDS category 2). Single SNP analysis of these five SNPs using FBAT showed that one SNP, rs12900948, was associated with decreased risk of developing neovascular AMD under a recessive genetic model (p=0.034)

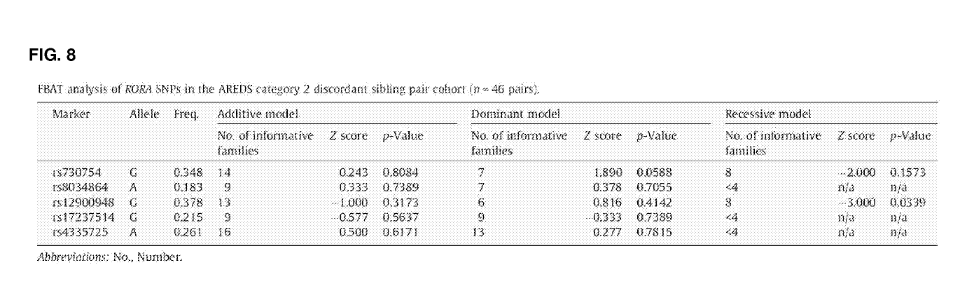

(FIG. 8). This SNP, like rs4335725, is also located in intron 1 of the RORA-001 transcript (ENST00000335670) and is also well conserved.

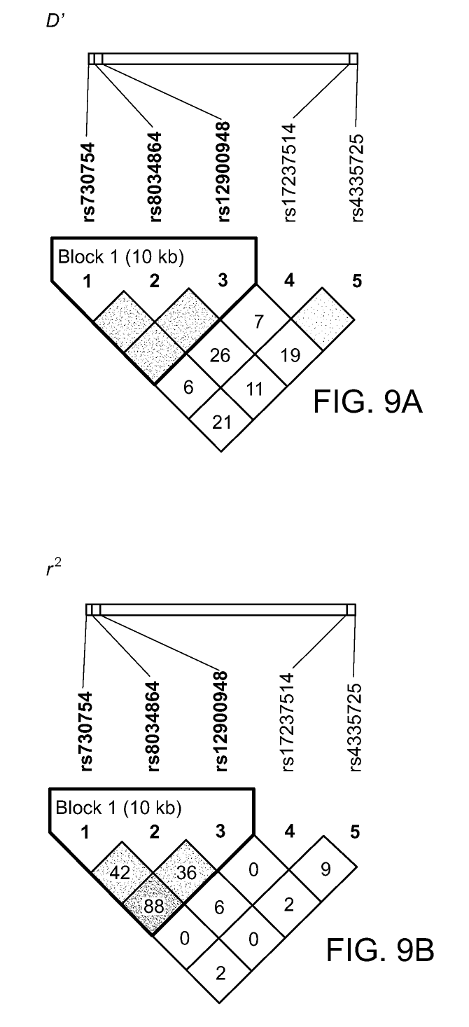

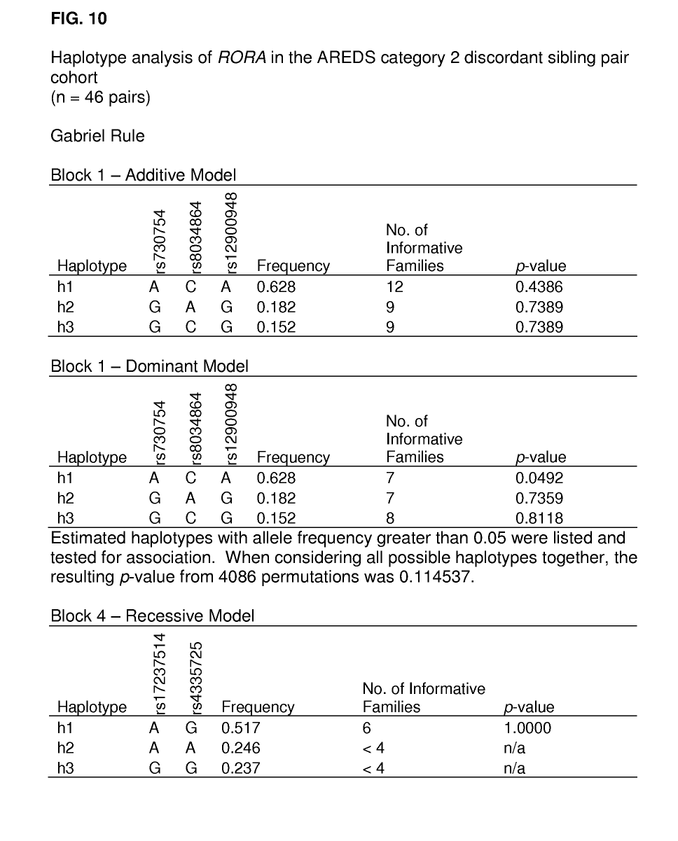

Haploview was used to create LD plots for this cohort (FIG. 9). Like the extremely discordant sibling pairs, applying the Gabriel rule demonstrated that block 1 containing SNPs rs730754, rs8034864 and rs12900948 was the same between both sibling pair cohorts studied (FIG. 5 and FIG. 9). However, the second haplotype block identified in the initial discovery cohort of extremely discordant sibling pairs was not found in the AREDS category 2 discordant sibling pair cohort. This may be due to the much smaller sample size analyzed (n=46 discordant sibling pairs). FBAT analysis of these two haplotype blocks in the AREDS category 2 discordant sibling pair cohort demonstrated that one haplotype (ACA) within this block was associated with neovascular AMD risk (p=0.0492). However, after permutation testing this finding was no longer significant (p=0.114) (FIG. 10).

F. Further Analysis of RORA SNPs and Haplotypes in the Family Patient Populations

Further analyses were also conducted on more than 500 sibling pairs as shown in Table 1. These analyses included sibling pairs where one sibling had neovascular AMD (the index sibling) and the other sibling (the control sibling) had intermediate AMD [AREDS category 3 (AREDS Research Group, 2000)] at 65 years of age were also recruited to the study. In addition, concordant sibling pairs were recruited for the study. Concordant sibling pairs qualified for the study if both members of the sibling pair had the neovascular form of AMD and are over 50 years of age. Individual SNPS were tested for association as described above.

TABLE 1

| SNP | Allele | Population | Effect | Model | p value |
|---|---|---|---|---|---|
| rs4335725 | A | AMD as Quantitative trait | Protective | Recessive | 0.0377 |
| rs4335725 | A | AMD as Quantitative trait (Adjusted) | Protective | Recessive | 0.0233 |
| rs4335725 | A | All AMD vs. Normal | Protective | Recessive | 0.0383 |
| rs4335725 | A | All AMD vs. Normal (Adjusted) | Protective | Recessive | 0.0204 |
| rs4335725 | A | Neovascular vs. Normal (Adjusted) | Protective | Recessive | 0.0295 |
| rs4335726 | A | AREDS 2 + 3 vs. Normal (Adjusted) | Protective | Recessive | 0.0320 |
| rs12900948 | G | Neovascular vs. Normal | Protective | Recessive | 0.0348 |
| rs12900948 | G | Neovascular vs. AREDS 2 + 3 | Protective | Recessive | 0.0440 |
| | | (Adjusted) | | | |

These results shown in Table 1 demonstrate that two SNPs, rs4335725 and rs12900948, are individually significant when comparing (i) siblings with neovascular AMD compared to normal siblings (AREDS category 1 or less) and (ii) siblings with neovascular AMD compared to siblings with early dry and intermediate AMD (AREDS categories 2 and 3). These two RORA SNPs remained significant after adjusting (i.e., controlling) for the following factors: CFH rs1061170 (Y402H), ARMS2/HTRA1 rs10490924/rs11200638, and smoking history in a multiple logistic regression model.

In addition, the results demonstrate that rs4335725 is significant when comparing all AMD populations to normal populations. Further, rs4335725 remained significant after controlling for the following factors: CFH rs1061170 (Y402H), ARMS2/HTRA1 rs10490924/rs11200638, and smoking history in a multiple logistic regression model.

Based on the family patient populations of more than 500 sibling pairs, haplotype blocks were constructed in Haploview (Gabriel, supra) and individual haplotypes were tested for association using FBAT as described above. These analyses confirmed the presence of two haplotype blocks as significantly associated with AMD. The results are shown in Tables 2 and 3 below.

TABLE 2

| rs12900948 | rs730754 | rs8034864 | | | | |
|---|---|---|---|---|---|---|
| (A > G) | (A > G) | (C > A) | Population | Effect | Model | p value |
| A | A | C | Neovascular | Risk | Additive | 0.05 |
| | | | vs. Normal | | | |
| A | A | C | Neovascular | Risk | Dominant | 0.006 |
| | | | vs. Normal | | | |
| A | A | C | Neovascular | Risk | Dominant | 0.04 |
| | | | vs. AREDS 2 | | | |
| A | A | C | Neovascular | Risk | Dominant | 0.03 |
| | | | vs. AREDS 2 | | | |
| | | | (Adjusted) | | | |

The results shown in Table 2 demonstrate that a haplotype comprising an adenine base at rs12900948, an adenine base at rs730754, and a cytosine base at rs8034864 indicates that a subject is at risk for developing AMD (i.e., a subject comprising this haplotype is more likely to develop AMD than a subject without this haplotype). This haplotype was determined to be significant in siblings with neovascular AMD compared to normal siblings (AREDS category 1 or less). In addition, this haplotype was determined to be significant in siblings with neovascular AMD compared to siblings with early dry (AREDS categories 2) and remained significant after adjusting for the following factors: CFH rs1061170 (Y402H), ARMS2/HTRA1 rs10490924/rs11200638, and smoking history in a multiple logistic regression model.

TABLE 3

| rs17237514 | rs4335725 | | | | |
|---|---|---|---|---|---|
| (A > G) | (G > A) | Population | Effect | Model | p value |
| A | A | AMD as Quantitative trait | Protective | Recessive | 0.02 |
| A | A | AMD as Quantitative trait (Adjusted) | Protective | Recessive | 0.01 |
| A | A | All AMD vs. Normal (Adjusted) | Protective | Recessive | 0.01 |
| A | A | Neovascular vs. Normal (Adjusted) | Protective | Recessive | 0.01 |
| A | A | AREDS 2 + 3 vs. Normal | Protective | Recessive | 0.01 |

The results shown in Table 3 demonstrate that a haplotype comprising an adenine base at rs17237514 and an adenine at rs433725 indicates that a subject is protected from developing AMD (i.e., a subject comprising this haplotype is less likely to develop AMD than a subject without this haplotype). This haplotype was determined to be significant in all AMD siblings when compared to normal siblings (AREDS category 1 or less) and remained significant after adjusting for the following factors: CFH rs1061170 (Y402H), ARMS2/HTRA1 rs10490924/rs11200638, and smoking history in a multiple logistic regression model.

G. Analysis of RORA SNPs and Haplotypes in the Greek Cohort

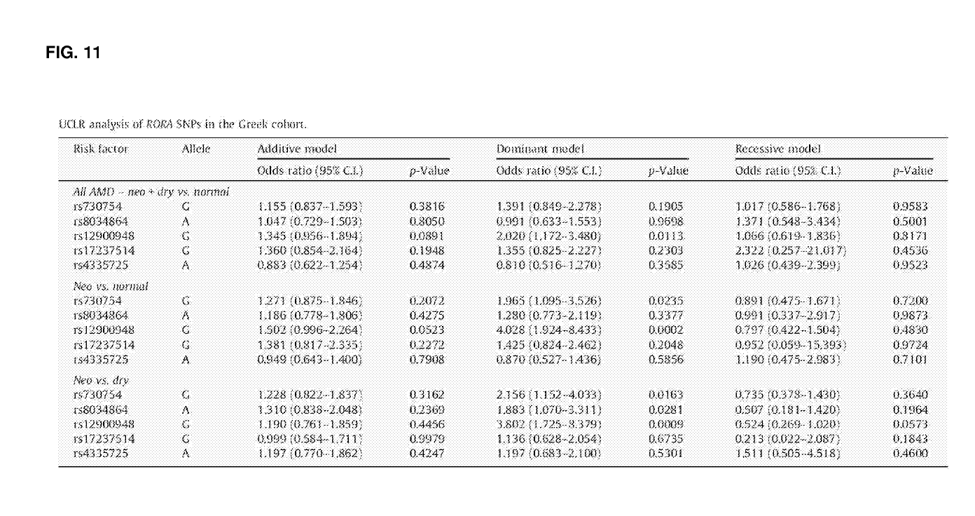

The SNPs that comprised the significant haplotypes (rs730754, rs8034864, rs12900948, for block 1 and rs17237514 and rs4335725 for block 4) in the discovery cohort were genotyped and tested for association in the Greek population (n=344) using unconditional logistic regression. This analysis showed that only rs12900948 was significantly associated with neovascular AMD when comparing neovascular AMD patients to normal patients and separately to patients with early and intermediate dry AMD (AREDS categories 2 and 3) under either a dominant or recessive model (FIG. 11). Specifically, the G allele (which is the minor allele for both family-based cohorts but the major allele for the Greek cohort) of rs12900948 increased risk of neovascular AMD in the unrelated case-control cohort by 4-fold and 3.8-fold when compared to normal patients and separately to patients with dry AMD respectively (OR: 4.028; 95% C.I.: 1.924, 8.433; p=0.0002 and OR: 3.802; 95% C.I.: 1.725, 8.379; p=0.0009). When the G allele of rs12900948 in normal controls (AREDS category 1 or less) is compared to all subtypes of AMD in this unrelated case control cohort, this finding was determined to be associated with AMD (OR: 2.020; 95% C.I.: 1.172, 3.480; p=0.0113).

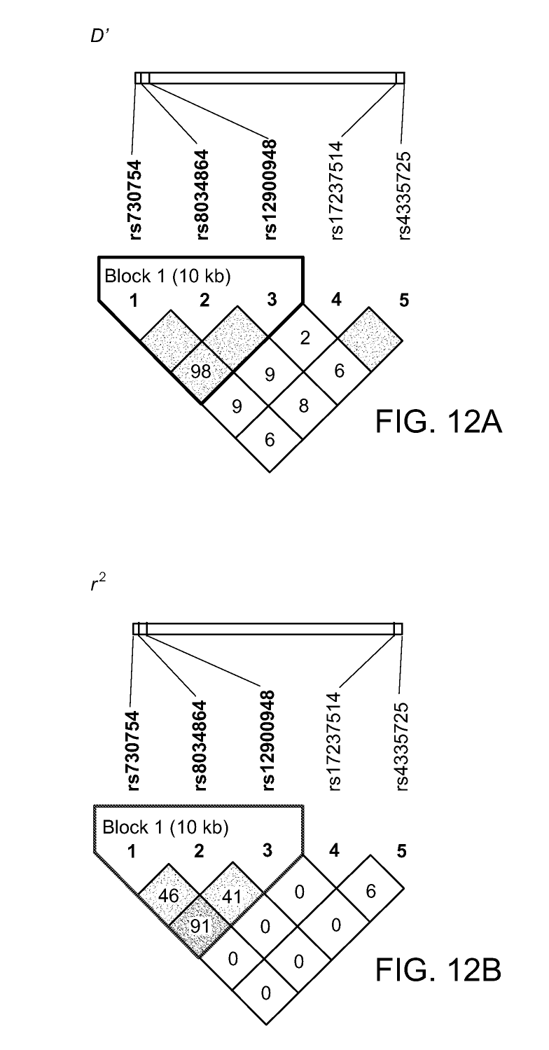

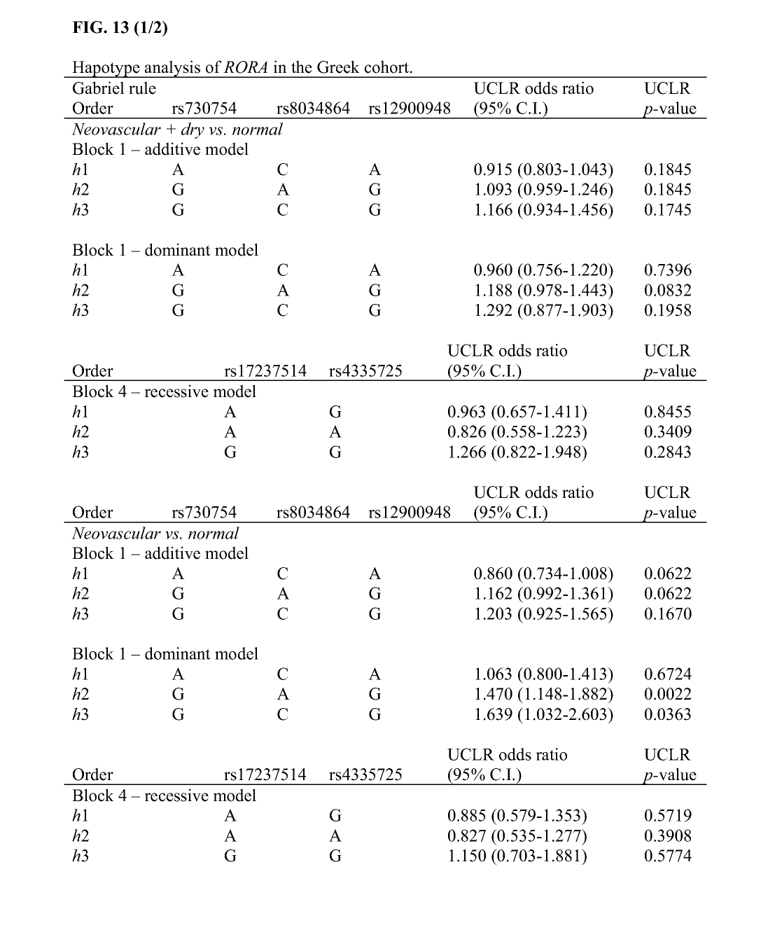

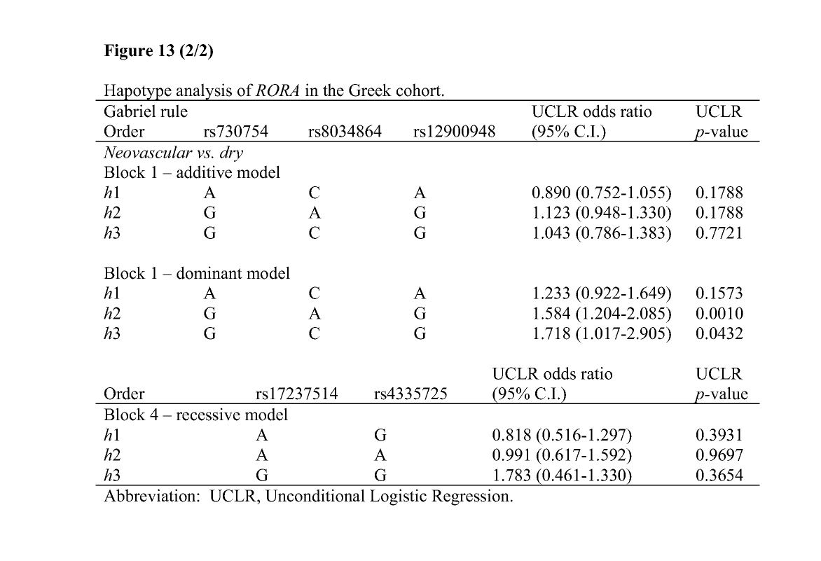

Haploview analysis using the Gabriel rule showed that there was a single haplotype block in the Greek unrelated case-control cohort (FIG. 12). This haplotype block, containing SNPs rs730754, rs8034864, rs12900948, was common to the cohort from Central Greece and both family-based cohorts studied (FIG. 5, FIG. 9, and FIG. 12). While SNPs rs17237514 and rs4335725 comprised a haplotype block for the initial discovery family-based cohort of extremely discordant sibling pairs, these SNPs did not comprise a second block (block 4) in the unrelated case-control cohort from Central Greece. Nonetheless, both blocks initially identified in the discovery cohort were tested in unconditional logistic regression in the unrelated case-control cohort. In the Greek population, the single haplotype block identified by the Gabriel rule in Haploview was significantly associated with AMD risk. Specifically, when either comparing neovascular patients to unaffected patients or separately, to dry patients, the h2 (GAG) haplotype was significantly associated with AMD risk under a dominant model (OR: 1.470; 95% C.I.: 1.148,1.882; p=0.0022 and OR: 1.584; 95% C.I.:1.204,2.085; p=0.0010, respectively) (FIG. 13). A second associated haplotype, h3 (GCG), in this same block (block 1), was also identified under a dominant model for both the comparison of neovascular patients to normal subjects and separately for neovascular patients compared to subjects with dry AMD (AREDS categories 2 and 3) (OR: 1.639; 95% C.I.: 1.032,2.603; p=0.0363 and OR: 1.718; 95% C.I.:1.01,2.905; p=0.0432, respectively).

H. Summary of Analysis of RORA SNPs and Haplotypes

The results described herein demonstrate an association between RORA and AMD. Using a family-based cohort comprised of extremely discordant sibling pairs, a single RORA SNP, rs4335725, and two haplotypes were identified significantly associated with neovascular AMD in FBAT. Study of a smaller discordant sibling population [where the index patient had neovascular AMD and the “control sibling” had a mild dry form of AMD (AREDS category 2)] identified a second SNP, rs12900948, as being associated with neovascular AMD. The rs12900948 SNP was part of a haplotype (GCG) that was identified as significantly associated with neovascular AMD risk in the initial discovery cohort of extremely discordant sibling pairs in FBAT. In a separate unrelated case control cohort from Central Greece, rs12900948 and two haplotypes containing this SNP (GAG and GCG) were also identified as associated with neovascular AMD when compared to either unrelated controls or to unrelated subjects with dry AMD (AREDS categories 2 and 3).

The single SNP rs12900948 was significant when comparing neovascular patients to patients with the dry form of AMD in both the family-based cohort and in the unrelated case control cohorts from Central Greece. This result suggests that the risk associated with rs129000948, or variants that are in LD with rs129000948, may specifically relate to the development of neovascular/advanced AMD.

In further studies where more than 500 sibling pairs were analyzed, rs12900948 was determined to be significant in sibling pairs where (i) one sibling had neovascular AMD and the other sibling was normal (AREDS category 1 or less) and (ii) one sibling had neovascular AMD and the other sibling had early dry or intermediate AMD (AREDS categories 2 and 3). In the family patient population, it was determined that the presence of a G at rs12900948 is protective (i.e., subjects with a G allele at rs12900948 are less likely to develop AMD than subjects without this allele). In this study, rs12900948 was also determined to be significant as part of a haplotype as shown in Table 2. In addition, r4335725 was determined to be significant in sibling pairs where (i) one sibling had neovascular AMD and the other sibling was normal (AREDS category 1 or less) and (ii) one sibling had neovascular AMD and the other sibling had early dry or intermediate AMD (AREDS categories 2 and 3). Further, rs4335725 was also determined to be significant as part of a haplotype as shown in Table 3.

The significant RORA variants and haplotypes associated with neovascular AMD in both the family-based and unrelated case control cohorts occur within the first intron of the RORA-001 transcript (ENST00000335670), a region that is well conserved across species, including mouse and rat. Although there is no apparent functional change, the results suggest that these or other undiscovered variations in intron 1, such as an insertion or deletion resulting in a change in copy number, may change the structure of the transcript and/or the sequence of unidentified modifying elements (e.g., silencers or enhancers) (Hastings et al. (2009) NAT. REV. GENET., 10, 551-564; Lupski (2007) NAT. GENET., 39, S43-S47). The disclosed variants may influence gene expression by altering modifying elements, increasing/decreasing RNA transcript stability, or affecting splicing with a resultant change in isoform expression (Hollams et al. (2002) NEUROCHEM. RES., 27, 957-980; Maddox et al. (2008) J. PHYSIOL., 586, 4409-4424; Margulies et al. (2008) NAT. REV. GENET., 9, 303-313).

However, analysis of the significant haplotypes suggest that the causal sequence change(s) in this region has not yet been identified. For example, the haplotype containing SNP rs4335725 was more significantly associated with risk of neovascular AMD than rs4335725 alone, suggesting that the causal variant(s) is likely in LD with this region. Additionally, SNP rs12900948 was significantly associated with neovascular AMD risk in the discovery cohort as part of a haplotype, but not individually in the discovery cohort.

Thesignificantly associated protective variants and protective and risk haplotypes described herein were identified in intron 1 region of RORA that has potential functional consequences as evidenced by the ENCODE Project (The ENCODE Project Consortium, Identification and analysis of functional elements in 1% of the human genome by the ENCODE pilot project. NATURE (2007) 447, 799-816), which suggests that intronic variants that are well-conserved across species (e.g., human, mouse, and rat) may be in genomic regions that contain silencers and enhancers. These changes may have postulated functional regulatory effects, suggesting possible etiologic mechanisms. For example, the G >A nucleotide change at rs4335725 may alter a binding site, thereby down regulating the RORA gene and decreasing risk of neovascular AMD as all significant haplotypes exert protective effects. Furthermore, it is becoming increasingly apparent that these well-conserved intronic variants may alter splice sites, thereby creating a new exon in the gene altogether (Maddox et al. (2008) J. PHYSIOLOGY 586:4409-4424). Moreover, in vivo and in vitro studies discuss that RORA may regulate 1) cytokines, such as interleukin-6 (IL-6), interleukin-8 (IL-8), tumor necrosis factor-alpha (TNF-α); 2) cell adhesion molecules, such as vascular cell adhesion molecule (VCAM-1) and the intracellular adhesion molecule (ICAM-1); and 3) lipoproteins, such as high density lipoprotein (HDL), serum amyloid A, and apolipoprotein Al (Lau et al. (2008) J. BIOL. CHEM. 283(26):18411; Migita et al. (2004) FEBS LETT. 557(1-3):269; Voyiaziakis et al. (1998) J. LIPID RES. 39(2):313). Thus, RORA may exerts its influence directly or indirectly via one or more of these mechanisms.

### Example 2

### Interaction of RORA SNPs with CFH and ARMS2/HTRA1

Using conditional logistic regression, interactions between RORA and CFH, ARMS2/HTRA1 and smoking were tested in the discovery cohort of 150 extremely discordant sibling pairs. No significant interaction was found between RORA and CFH and separately between RORA and smoking history. A significant interaction was found between the RORA SNP rs12900948 and the ARMS2/HTRA1 SNPs rs10490924, rs11200638, and rs1049331 (p=0.0044, 0.0044, and 0.0038 respectively). Based on this data, HTRA1 was incorporated into the network pathway analysis that included RORA using IPA's “Path Designer” function to explore a hypothetical molecular means of interaction. The addition of HTRA1 introduced the following genes: bone morphogenetic protein 4 (BMP4), family with sequence similarity 46, member A (FAM46A), fibronectin 1 (FN1), growth differentiation factor 5 (GDF5), interleukin 13 (IL13), matrix metallopeptidase 3 (MMP3), matrix metallopeptidase 1 (MMP1), serpin peptidase inhibitor, clade A, member 1 (SERPINA1), transforming growth factor, beta 2 (TGFb2), transforming growth factor, beta 3 (TGFb3) into the analysis. ARMS2 was not included in network analysis as it was not available by IPA's database.

In this study, a statistically significant interaction between RORA and the ARMS2/HTRA1 locus was identified. This finding was prospectively validated in two nested case-control cohorts (D. Schaumberg et al., paper under review). To date, the ARMS2/HTRA1 locus is the region most significantly associated with neovascular AMD (Fisher, et al. (2005) HUM. MOL. GENET., 14, 2257-2264; Shuler, et al. (2007) ARCH. OPHTHALMOL., 125, 63-67; Zhang, et al. (2008) BMC MED. GENET., 9, 51), however, the pathway in which it functions has yet to be elucidated. This interaction suggests that RORA may be functioning in a similar, an overlapping, or the same pathway as ARMS2/HTRA1.

### Example 3

### Expression Quantitative Trait Loci (eQTL) Analysis

Another potential result of genetic variation(s) in RORA is the cis or trans regulation of a nearby locus, a distant gene, or the indirect regulation of a gene by means of influencing a regulatory locus (for review please see Ioannidis, et al. (2009) NAT. REV. GENET., 10, 318-329). In any of these scenarios, variation within RORA could affect transcription of another gene(s) that in turn would influence the development and progression of AMD. This example describes our investigation of the association of the RORA SNPs found to be significant in our study (either directly or those within a significant haplotype) with expression quantitative trait loci (eQTL) analysis.

Using the publicly available expression quantitative trait loci (eQTL) database mRNA by SNP Browser v 1.0.1 (Dixon, et al. (2007) NAT. GENET., 39, 1202-1207; Moffatt et al. (2007) NATURE 448, 470-473; Shimada, et al. (2009) NUCL. ACIDS RES., 37, D810-D815), the association between eQTLs and the RORA SNPs that were identified as significant either individually or as part of a haplotype were investigated.

Increasingly, data from GWAS and microarray studies have been analyzed in combination to uncover expression quantitative trait loci (eQTL) that relate specific SNPs to global or tissue specific expression of gene transcripts (Cookson et al. (2009) NAT. REV. GENET., 10:184-194). Using the publicly available eQTL database mRNA by SNP Browser v 1.0.1 (Dixon, et al. (2007) NAT. GENET., 39, 1202-1207; Moffatt et al., supra), the association of the RORA SNPs (i.e., the RORA SNPs that comprised the haplotypes described herein) with eQTLs was investigated. Of the five SNPs discussed herein, rs8034864, rs730754, and rs4335725 were present in the database, but none were significantly associated with transcript expression according to the method for calculating significance defined by Dixon et al., supra. The interaction between the RORA SNP rs12900948 and the ARMS2/HTRA1 SNPs led us to investigate the association of these SNPs, rs10490924, rs11200638, and rs1049331, with transcript expression. Of the ARMS2/HTRA1 SNPs, only rs10490924 was present in the database, but was not significantly associated with the expression of any transcripts.

Based on the five SNPs investigated in this study, only rs8034864, rs730754, and rs4335725 were present in the database. None of these three SNPs were significantly associated with transcript expression. This result is consistent with our haplotype analysis demonstrating that these SNPs are likely not individually causal.

### Example 4

### Genotyping of Microsatellite Markers

For the genotyping of microsatellite markers, the Sequenom iPLEX system technology, and direct sequencing protocols, leukocyte DNA was either purified by using standard phenol-chloroform or DNAzol (Invitrogen) extraction protocols. Using 133 extremely discordant sibling pairs, 18 highly heterozygous microsatellite markers spanning 34 megabases of the 15q21-22 region (FIG. 2) were analyzed. These markers included several that were in the vicinity of RORA. All markers were fluorescently labeled with either 5-carboxyfluorescein or 6-carboxyfluorescein on the 50 end of the reverse primer and an additional sequence of CTGTCTT was added to the 50 end of the forward primer. PCR was used to amplify genomic DNA fragments from 20 ng of leukocyte DNA in a solution of 10× PCR buffer containing 25 mmol/L of MgCl2; 0.2 mmol/L each of deoxyadenosine triphosphate, deoxythymidine triphosphate, deoxyguanosine triphosphate, and deoxycytidine triphosphate; and 0.5 U of Taq DNA polymerase (USB Corporation, Cleveland, Ohio). PCR cycling conditions were as follows, 95° C. for 5 min, followed by 35 cycles of 54-60° C. (specific to primer pair) for 30 s, 72° C. for 30 s, and 95° C. for 30 s, with a final annealing at 54-60° C. (specific to primer pair) for 1.5 min and extension of 72° C. for 5 min Polymerase chain reaction products were diluted 1:20 for markers labeled with FAM and 1:10 for markers labeled with HEX. Samples were pooled according to product size and denatured before being genotyped on the ABI 3730×1 DNA Analyzer (Applied Biosystems). Data were then analyzed using ABI's Genemapper 3.7 software, which interrogates the quality of the size standard and makes the appropriate genotype calls based on size. For quality control purposes, all genotypes were then evaluated manually.

For linkage analysis of the 18 microsatellite markers, identity-by-state (IBS) scores were calculated from the number of alleles (0, 1 or 2) shared between each pair, the index and the discordant sibling, for each of the 18 markers. Using heterozygosities for each marker obtained from Map-O-Mat (available at www.compgen.rutgers.edu/mapomat), the expected IBS (null hypothesis of no linkage) was calculated and then compared with the observed IBS values. A goodness of fit test was applied to assess the significance of the difference between the observed and expected distribution. This method has been used previously for linkage analysis in sibling pairs (DeAngelis, et al. (2008) OPHTHALMOL, 115, 1209-1215).

Calculation of identity-by-state scores from the genotyping results of 18 highly heterozygous microsatellite markers in the 15q region identified three markers, D15S1015, D155209, and D155214, that were associated with neovascular AMD (p<0.05) (FIG. 4). The most significantly associated marker, D15S117 (p=0.01), is located approximately 2 megabases from the gene end (RORA: ENSG00000069667) (FIG. 2).

### Example 5

### Diagnosis and Prognosis of AMD

It is contemplated that the skilled artisan can carry out diagnostic or prognostic methods according to the invention by detecting (directly or indirectly) the presence of absence of one or more of the polymorphic variants located, for example, at rs12900948 and rs4335725 (including detecting a proxy polymorphic variant at a proxy polymorphic site) or a haplotype defined by the polymorphic sites rs4335725 and rs17237514 or the polymorphic sites rs12900948, rs730754, and rs8034864. For example, any of the detection techniques described herein can be used. It is further contemplated that individuals with one or more protective variants or a protective haplotype, as identified above, will have a lower risk of developing neovascular AMD as compared to those without the protective variants or protective haplotype. It is also further contemplated that individuals with one or more risk variants or a risk haplotype, as identified above, will have a greater risk of developing neovascular AMD as compared to those without the risk variants or risk haplotype.

### INCORPORATION BY REFERENCE

The entire content of each patent and non-patent document disclosed herein is expressly incorporated herein by reference for all purposes, including Silveira et al., (2010) VISION RESEARCH 50(7):698-715.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method for determining a subject's risk of developing age-related macular degeneration, the method comprising detecting in a sample from the subject the presence or absence of an allelic variant at a polymorphic site in the RORA gene that is associated with risk of developing age-related macular degeneration.

2. The method of claim 1, comprising detecting the presence or absence of a protective variant at a polymorphic site in the RORA gene, wherein, if the subject has the protective variant, the subject is less likely to develop age-related macular degeneration than a person without the protective variant.

3. The method of claim 1, wherein the polymorphic site comprises a site selected from the group consisting of rs12900948 and rs4335725.

4. The method of claim 3, wherein the polymorphic site is rs12900948.

5. The method of claim 4, wherein for the rs12900948 polymorphic site, the forward sequence comprises GAGTCTTTCTGATGGTGAGC[X₅]GGGTGATGCCATAACCCGGG (SEQ ID NO. 5) wherein X₅ is an adenine to a guanine substitution and/or the reverse sequence comprises CCCGGGTTATGGCATCACCC[X₆]GCTCACCATCAGAAAGACTC (SEQ ID NO. 6) wherein X₆ is a thymine to a cytosine substitution.

6. The method of claim 4 any one of the proceeding claims, comprising detecting a guanine or adenine base at rs12900948.

7. The method of claim 3, wherein the polymorphic site is rs4335725.

8. The method of claim 7, wherein for the rs4335725 polymorphic site, the forward sequence comprises GCCTTCCAGAAGTGACTTCT[X₁₅]TAACTCATTTGTAAATGTTG (SEQ ID NO. 15) wherein X₁₅ is a guanine to an adenine substitution and/or the reverse sequence comprises CAACATTTACAAATGAGTTA[X₁₆]AGAAGTCACTTCTGGAAGGC (SEQ ID NO. 16) wherein X₁₆ is a cytosine to a thymine substitution.

9. The method of claim 7, comprising detecting an adenine or a guanine base at rs4335725.

10. The method of claim 1, wherein the allelic variant defines a haplotype.

11. The method of claim 1, wherein the detecting step comprises direct nucleotide sequencing.

12. The method of claim 1, wherein the detecting step comprises hybridization using a hybridization probe that selectively anneals to the variant allele or to the common allele at the polymorphic site of the RORA gene.

13. The method of claim 1, wherein the detecting step comprises restriction fragment length polymorphism analysis.

14. The method of claim 1, further comprising the step of amplifying the polymorphic site prior to the detecting step.

15. The method of claim 1, wherein the detecting step comprises an amplification reaction using primers capable of amplifying the polymorphic site.

16. A method of determining a subject's risk of developing age-related macular degeneration, the method comprising detecting in a sample from a subject the presence or absence of a haplotype in the RORA gene, wherein the haplotype is selected from the group consisting of:
(a) a risk haplotype that is more frequently present in individuals diagnosed with AMD compared to healthy individuals, or
(b) a protective haplotype that is more frequently present in healthy individuals compared to individuals diagnosed with AMD.

17. The method of claim 16, wherein the haplotype is defined by the alleles present at rs12900948, rs730754, and rs8034864.

18. The method of claim 17, comprising detecting an adenine or guanine base at rs12900948, an adenine or guanine base at rs730754, and an cytosine or adenine base at rs8034864.

19. The method of claim 16 or 17, wherein the haplotype is a risk haplotype comprising an adenine in the forward sequence of rs12900948, an adenine in the forward sequence of rs730754, and a cytosine in the forward sequence of rs8034864.

20. The method of claim 16, wherein the haplotype is defined by the alleles present at rs17237514 and rs4335725.

21. The method of claim 20, comprising detecting an adenine or guanine base at rs17237514 and an adenine or guanine base at rs4335725.

22. The method of claim 20, wherein haplotype is a protective haplotype comprising an adenine in the forward sequence of rs17237514 and an adenine in the forward sequence of rs4335725.

23. The method of claim 16, wherein the detecting step comprises direct nucleotide sequencing.

24. The method of claim 16, wherein the detecting step comprises hybridization using a hybridization probe that selectively anneals to the variant allele or to the common allele at the polymorphic site of the RORA gene.

25. The method of claim 16, wherein the detecting step comprises restriction fragment length polymorphism analysis.

26. The method of claim 23, further comprising the step of amplifying the polymorphic site prior to the detecting step.

27. The method of claim 16, wherein the detecting step comprises an amplification reaction using primers capable of amplifying the polymorphic site.

28-29. (canceled)

30. A device or kit for diagnosing susceptibility to age-related macular degeneration (AMD) in a subject comprising oligonucleotides that distinguish alleles at at least one polymorphic site in the RORA gene associated with risk of developing AMD.

31. The device or kit of claim 30, wherein the oligonucleotides distinguish alleles at at least one polymorphic site selected from the group consisting of rs12900948, rs730754, rs8034864, rs17237514 and rs4335725.

32. The device or kit of claim 30, wherein the oligonucleotides are primers for nucleic acid amplification of a region spanning a RORA gene polymorphic site selected from the group consisting of rs12900948, rs730754, rs8034864, rs17237514 and rs4335725.

33. The device or kit of claim 30, wherein the oligonucleotides are probes for nucleic acid hybridization of a region spanning a RORA gene polymorphic site selected from the group consisting of rs12900948, rs730754, rs8034864, rs17237514 and rs4335725.
